(19) United States
(12) Patent Application Publication
(10) Pub. No.: US 2007/0073059 A1
(43) Pub. Date: Mar. 29, 2007

(54) Method of producing azomethine dye compound

(75) Inventors: Keiza Kimura, Odawara-shi (JP); Katsuyoshi Yamakawa, Odawara-shi (JP); Osamu Yonekura, Odawara-shi (JP)

Correspondence Address: BIRCH STEWART KOLASCH & BIRCH, PO BOX 747, FALLS CHURCH, VA, 22040-0747, US

(73) Assignee: FUJI PHOTO FILM CO., LTD.

(21) Appl. No.: 11/528,468

(22) Filed: Sep. 28, 2006

(30) Foreign Application Priority Data
Sep. 29, 2005 (JP) 2005-285310

(51) Int. Cl. C07D 487/04

(52) U.S. Cl. 544/238; 546/272.4; 548/262.4

(57) ABSTRACT

A method of producing a compound represented by formula (I), including reacting a compound represented by formula (II) with a compound represented by formula (III): 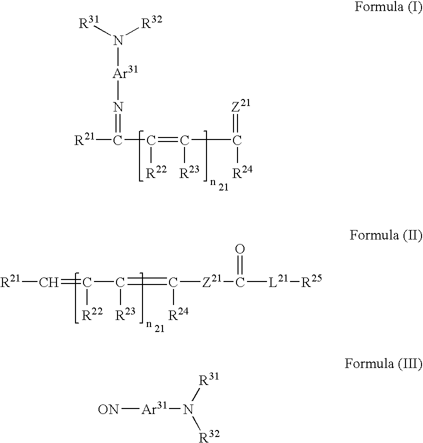
wherein, in formulas (I) to (III), Ar³¹ represents a divalent aromatic group or a divalent heterocyclic group; L²¹ represents a single bond, —O—, or —N(R²⁶)—; Z²¹ represents —O— or —N(R²⁷)—; R²¹, R²², R²³, R²⁴, and R²⁷ each independently represent a hydrogen atom or a substituent; R²⁵, R²⁶, R³¹, and R³² each independently represent a hydrogen atom, an aliphatic group, an aromatic group, or a heterocyclic group bonding through its carbon atom; and n₂₁ represents 0 or 1, wherein any of R²¹, R²², R²³, R²⁴, and R²⁷ may bond to one another to form a ring, and any of Ar³¹, R³¹, and R³² may bond to one another to form a ring.

### FIELD OF THE INVENTION

The present invention relates to a method of producing azomethine dye compounds useful as image-forming materials for printing such as inkjet printing and thermal transfer printing; optical recording element materials, optical film materials, and the like. More specifically, the present invention relates to a method of producing azomethine dye compounds in high yield under mild conditions without need of complicated operations.

### BACKGROUND OF THE INVENTION

Azomethine dyes have a variety of colors such as yellow, red, magenta, blue, and cyan and thus are widely used as image-forming dyes particularly in the subtractive color process. Specifically, azomethine dyes formed by oxidative coupling of a 5,5-condensed-ring type heterocyclic compound and p-dialkylaminoaniline are characterized by having a sharp hue and good color reproducibility. It is known that conventional azomethine dyes are formed by an oxidative coupling reaction of an active methylene compound or a phenol compound with an N,N-dialkyl-p-phenylenediamine compound in a water-containing solvent system or a two-phase system of water and an organic solvent (for example, see JP-A-60-32851 (“JP-A” means unexamined published Japanese patent application) and Journal of the American Chemical Society, Vol. 79, p. 2919 (1957)). These reaction systems are not efficient, because the oxide of the N,N-dialkyl-p-phenylenediamine compound undergoes hydrolysis in these reaction systems. Thus, there have been proposed improved methods using nonaqueous-solvent systems. In such methods, however, operation is complicated in light of mass production, because such methods require that the starting materials be added in parts so as not to be decomposed (for example, see JP-A-4-126772). Dehydration condensation reactions using p-dialkylaminonitrosobenzene in place of the N,N-dialkyl-p-phenylenediamine compound are also known (for example, see JP-A-4-178646). All of these methods involve complicated operation or give insufficient yield, and thus improvements of these methods have been demanded.

### SUMMARY OF THE INVENTION

The present invention resides in a method of producing a compound represented by formula (I), comprising reacting a compound represented by formula (II) with a compound represented by formula (III):

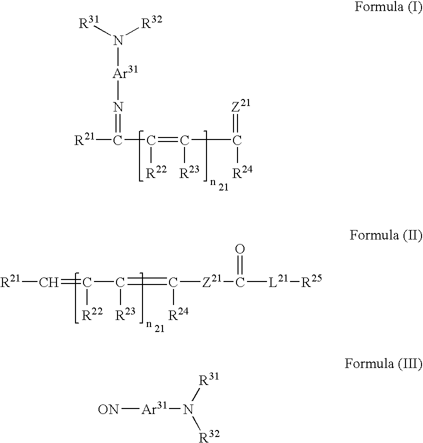

wherein, in formulas (I) to (III), Ar³¹ represents a divalent aromatic group or a divalent heterocyclic group; L²¹ represents a single bond, —O—, or —N(R²⁶)—; Z²¹ represents —O— or —N(R²⁷)—; R ²¹, R²², R²³, R²⁴, and R²⁷ each independently represent a hydrogen atom or a substituent; R²⁵, R²⁶, R³¹, and R³² each independently represent a hydrogen atom, an aliphatic group, an aromatic group, or a heterocyclic group bonding through its carbon atom; and n₂₁ represents 0 or 1, wherein any of R ²¹, R ²², R²³, R²⁴, and R²⁷ may bond to one another to form a ring, and any of Ar³¹, R³¹, and R³² may bond to one another to form a ring.

Other and further features and advantages of the invention will appear more fully from the following description.

### DETAILED DESCRIPTION OF THE INVENTION

As a result of active investigations, the present inventors have found that the above-mentioned problems can be overcome by the method as described below and have completed the present invention. Thus, the present invention provides:

(1) A method of producing a compound represented by formula (I), comprising reacting a compound represented by formula (II) with a compound represented by formula (III):

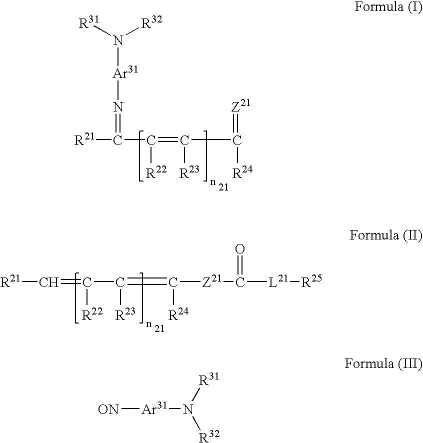

wherein, in formulas (I) to (III), Ar³¹ represents a divalent aromatic group or a divalent heterocyclic group; L²¹ represents a single bond, —O—, or —N(R²⁶)—; Z²¹ represents —O— or —N(R²⁷)—; R²¹, R²², R²³, R²⁴, and R²⁷ each independently represent a hydrogen atom or a substituent; R²⁵, R²⁶, R³¹, and R³² each independently represent a hydrogen atom, an aliphatic group, an aromatic group, or a heterocyclic group bonding through its carbon atom; and n₂₁ represents 0 or 1, wherein any of R²¹, R²², R²³, R²⁴, and R²⁷ may bond to one another to form a ring, and any of Ar³¹, R³¹, and R³² may bond to one another to form a ring;

(2) The method according to Item (1), wherein the compound represented by formula (I) is a compound represented by formula (I-1) or (I-2), the compound represented by formula (II) is a compound represented by formula (II-1) or (II-2), and the compound represented by formula (III) is a compound represented by formula (III-1):

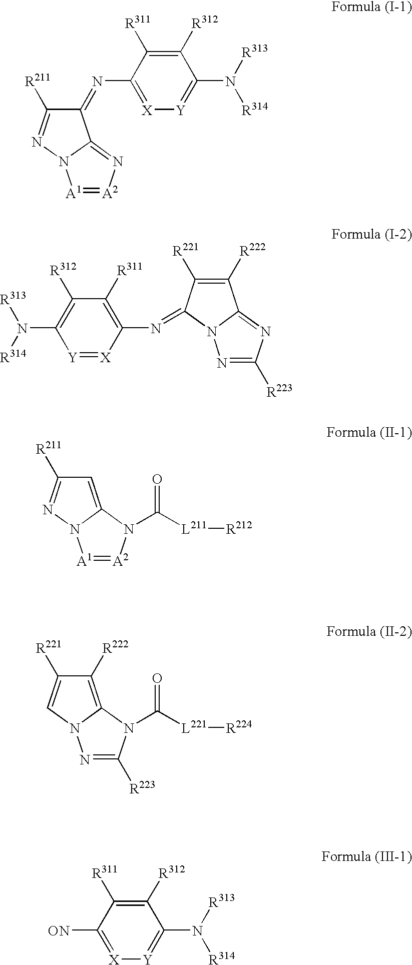

wherein, in formulas (I-1), (I-2), (II-1), (II-2), and (III-1), L²¹¹ and L²²¹ each independently represent a single bond, —O—, or —N(R²¹³)—; one of A¹ and A² represents a nitrogen atom and the other represents —C(R²¹⁴)═; X and Y each independently represent a nitrogen atom or ═C(R³¹⁵)—; R²¹¹, R²¹⁴, R²²¹, R²²², R²²³, R³¹¹, R³¹², and R³¹⁵ each independently represent a hydrogen atom or a substituent; and R²¹², R²¹³, R²²⁴, R³¹³, and R³¹⁴ each independently represent a hydrogen atom, an aliphatic group, an aromatic group, or a heterocyclic group bonding through its carbon atom;

(3) The method according to Item (1) or (2), wherein the reaction is carried out in the presence of a heterocyclic compound containing at least one nitrogen atom, and the heterocyclic compound differs from the compounds represented by formulas (I) to (III);

(4) The method according to any one of Items (1) to (3), wherein the reaction is carried out in the presence of a dehydrating condensing agent; and

(5) The method according to any one of Items (1) to (4), wherein the method produces a dye compound.

According to the production method of the present invention, azomethine dye compounds useful as image-forming materials for printing, such as inkjet printing and thermal transfer printing; optical recording element materials, optical film materials, and the like can be produced in high yield under mild conditions without use of complicated operations.

The modes for carrying out the present invention are explained in detail below.

In the present specification, the aliphatic group means an alkyl group, a substituted alkyl group, an alkenyl group, a substituted alkenyl group, an alkynyl group, a substituted alkynyl group, an aralkyl group, and a substituted aralkyl group. The aforementioned alkyl group may have a branch or may form a ring (i.e. a cycloalkyl group). The alkyl group preferably has 1 to 20 carbon atoms, and more preferably 1 to 18 carbon atoms. The alkyl moiety in the aforementioned substituted alkyl group is the same as the above mentioned alkyl group. The aforementioned alkenyl group may have a branch or may form a ring (i.e. a cycloalkenyl group). The alkenyl group has preferably 2 to 20 carbon atoms, and more preferably 2 to 18 carbon atoms. The alkenyl moiety in the aforementioned substituted alkenyl group is the same as the above mentioned alkenyl group. The aforementioned alkynyl group may have a branch or may form a ring. (i.e. a cycloalkynyl group) The alkynyl group has preferably 2 to 20 carbon atoms, and more preferably 2 to 18 carbon atoms. The alkynyl moiety in the aforementioned substituted alkynyl group is the same as the above mentioned alkynyl group. The alkyl moiety in the aforementioned aralkyl group and substituted aralkyl group is the same as the above mentioned alkyl group. The aryl moiety in the aforementioned aralkyl group and substituted aralkyl group is the same as the below mentioned aryl group.

Specific examples of the substituent in the substituted alkyl group, the substituted alkenyl group, the substituted alkynyl group, and the alkyl moiety in the substituted aralkyl group include: a halogen atom (e.g. a chlorine atom, a bromine atom, or an iodine atom); an alkyl group [which represents a substituted or unsubstituted linear, branched, or cyclic alkyl group, and which includes an alkyl group (preferably an alkyl group having 1 to 30 carbon atoms, e.g. a methyl group, an ethyl group, an n-propyl group, an isopropyl group, a t-butyl group, an n-octyl group, an eicosyl group, a 2-chloroethyl group, a 2-cyanoethyl group, or a 2-ethylhexyl group), a cycloalkyl group (preferably a substituted or unsubstituted cycloalkyl group having 3 to 30 carbon atoms, e.g. a cyclohexyl group, a cyclopentyl group, or a 4-n-dodecylcyclohexyl group), a bicycloalkyl group (preferably a substituted or unsubstituted bicycloalkyl group having 5 to 30 carbon atoms, i.e. a monovalent group obtained by removing one hydrogen atom from a bicycloalkane having 5 to 30 carbon atoms, e.g. a bicyclo[1,2,2]heptan-2-yl group or a bicyclo[2,2,2]octan-3-yl group), and a tricyclo or higher structure having three or more ring structures; and an alkyl group in a substituent explained below (e.g. an alkyl group in an alkylthio group) represents such an alkyl group of the above concept]; an alkenyl group [which represents a substituted or unsubstituted linear, branched, or cyclic alkenyl group, and which includes an alkenyl group (preferably a substituted or unsubstituted alkenyl group having 2 to 30 carbon atoms, e.g. a vinyl group, an allyl group, a prenyl group, a geranyl group, or an oleyl group), a cycloalkenyl group (preferably a substituted or unsubstituted cycloalkenyl group having 3 to 30 carbon atoms, i.e. a monovalent group obtained by removing one hydrogen atom from a cycloalkene having 3 to 30 carbon atoms, e.g. a 2-cyclopenten-1-yl group or a 2-cyclohexen-1-yl group), and a bicycloalkenyl group (which represents a substituted or unsubstituted bicycloalkenyl group, preferably a substituted or unsubstituted bicycloalkenyl group having 5 to 30 carbon atoms, i.e. a monovalent group obtained by removing one hydrogen atom from a bicycloalkene having one double bond, e.g. a bicyclo[2,2,1]hept-2-en-1-yl group or a bicyclo[2,2,2]oct-2-en-4-yl group)]; an alkynyl group (preferably a substituted or unsubstituted alkynyl group having 2 to 30 carbon atoms, e.g. an ethynyl group, a propargyl group, or a trimethylsilylethynyl group); an aryl group (preferably a substituted or unsubstituted aryl group having 6 to 30 carbon atoms, e.g. a phenyl group, a p-tolyl group, a naphthyl group, an m-chlorophenyl group, or an o-hexadecanoylaminophenyl group); a heterocyclic group (preferably a monovalent group obtained by removing one hydrogen atom from a substituted or unsubstituted 5- or 6-membered aromatic or nonaromatic heterocyclic compound; more preferably a 5- or 6-membered aromatic heterocyclic group having 3 to 30 carbon atoms, e.g. a 2-furyl group, a 2-thienyl group, a 2-pyrimidinyl group, a 2-benzothiazolyl group); a cyano group; a hydroxyl group; a nitro group; a carboxyl group; an alkoxy group (preferably a substituted or unsubstituted alkoxy group having 1 to 30 carbon atoms, e.g. a methoxy group, an ethoxy group, an isopropoxy group, a t-butoxy group, an n-octyloxy group, or a 2-methoxyethoxy group); an aryloxy group (preferably a substituted or unsubstituted aryloxy group having 6 to 30 carbon atoms, e.g. a phenoxy group, a 2-methylphenoxy group, a 4-t-butylphenoxy group, a 3-nitrophenoxy group, or a 2-tetradecanoylaminophenoxy group); a silyloxy group (preferably a silyloxy group having 3 to 20 carbon atoms, e.g. a trimethylsilyloxy group or a t-butyldimethylsilyloxy group); a heterocyclic oxy group (preferably a substituted or unsubstituted heterocyclic oxy group having 2 to 30 carbon atoms, e.g. a 1 -phenyltetrazol-5-oxy group or a 2-tetrahydropyranyloxy group); an acyloxy group (preferably a formyloxy group, a substituted or unsubstituted alkylcarbonyloxy group having 2 to 30 carbon atoms, or a substituted or unsubstituted arylcarbonyloxy group having 7 to 30 carbon atoms, e.g. a formyloxy group, an acetyloxy group, a pivaloyloxy group, a stearoyloxy group, a benzoyloxy group, or a p-methoxyphenylcarbonyloxy group); a carbamoyloxy group (preferably a substituted or unsubstituted carbamoyloxy group having 1 to 30 carbon atoms, e.g. an N,N-dimethylcarbamoyloxy group, an N,N-diethylcarbamoyloxy group, a morpholinocarbonyloxy group, an N,N-di-n-octylaminocarbonyloxy group, or an N-n-octylcarbamoyloxy group); an alkoxycarbonyloxy group (preferably a substituted or unsubstituted alkoxycarbonyloxy group having 2 to 30 carbon atoms, e.g. a methoxycarbonyloxy group, an ethoxycarbonyloxy group, a t-butoxycarbonyloxy group, or an n-octylcarbonyloxy group); an aryloxycarbonyloxy group (preferably a substituted or unsubstituted aryloxycarbonyloxy group having 7 to 30 carbon atoms, e.g. a phenoxycarbonyloxy group, a p-methoxyphenoxycarbonyloxy group, or a p-n-hexadecyloxyphenoxycarbonyloxy group); an amino group (preferably an amino group, a substituted or unsubstituted alkylamino group having 1 to 30 carbon atoms, or a substituted or unsubstituted anilino group having 6 to 30 carbon atoms, e.g. an amino group, a methylamino group, a dimethylamino group, an anilino group, an N-methyl-anilino group, or a diphenylamino group); an acylamino group (preferably a formylamino group, a substituted or unsubstituted alkylcarbonylamino group having 1 to 30 carbon atoms, or a substituted or unsubstituted arylcarbonylamino group having 6 to 30 carbon atoms, e.g. a formylamino group, an acetylamino group, a pivaloylamino group, a lauroylamino group, a benzoylamino group, or a 3,4,5-tri-n-octyloxyphenylcarbonylamino group); an aminocarbonylamino group (preferably a substituted or unsubstituted aminocarbonylamino group having 1 to 30 carbon atoms, e.g. a carbamoylamino group, an N,N-dimethylaminocarbonylamino group, an N,N-diethylaminocarbonylamino group, or a morpholinocarbonylamino group); an alkoxycarbonylamino group (preferably a substituted or unsubstituted alkoxycarbonylamino group having 2 to 30 carbon atoms, e.g. a methoxycarbonylamino group, an ethoxycarbonylamino group, a t-butoxycarbonylamino group, an n-octadecyloxycarbonylamino group, or an N-methyl-methoxycarbonylamino group); an aryloxycarbonylamino group (preferably a substituted or unsubstituted aryloxycarbonylamino group having 7 to 30 carbon atoms, e.g. a phenoxycarbonylamino group, a p-chlorophenoxycarbonylamino group, or an m-n-octyloxyphenoxycarbonylamino group); a sulfamoylamino group (preferably a substituted or unsubstituted sulfamoylamino group having 0 to 30 carbon atoms, e.g. a sulfamoylamino group, an N,N-dimethylaminosulfonylamino group, or an N-n-octylaminosulfonylamino group); an alkyl- or aryl-sulfonylamino group (preferably a substituted or unsubstituted alkylsulfonylamino group having 1 to 30 carbon atoms, or a substituted or unsubstituted arylsulfonylamino group having 6 to 30 carbon atoms, e.g. a methylsulfonylamino group, a butylsulfonylamino group, a phenylsulfonylamino group, a 2,3,5-trichlorophenylsulfonylamino group, or a p-methylphenylsulfonylamino group); a mercapto group; an alkylthio group (preferably a substituted or unsubstituted alkylthio group having 1 to 30 carbon atoms, e.g. a methylthio group, an ethylthio group, or an n-hexadecylthio group); an arylthio group (preferably a substituted or unsubstituted arylthio group having 6 to 30 carbon atoms, e.g. a phenylthio group, a p-chlorophenylthio group, or an m-methoxyphenylthio group); a heterocyclic thio group (preferably a substituted or unsubstituted heterocyclic thio group having 2 to 30 carbon atoms, e.g. a 2-benzothiazolylthio group or a 1-phenyltetrazol-5-ylthio group); a sulfamoyl group (preferably a substituted or unsubstituted sulfamoyl group having 0 to 30 carbon atoms, e.g. an N-ethylsulfamoyl group, an N-(3-dodecyloxypropyl)sulfamoyl group, an N,N-dimethylsulfamoyl group, an N-acetylsulfamoyl group, an N-benzoylsulfamoly group, or an N-(N′-phenylcarbamoyl)sulfamoyl group); a sulfo group; an alkyl- or aryl-sulfinyl group (preferably a substituted or unsubstituted alkylsulfinyl group having 1 to 30 carbon atoms, or a substituted or unsubstituted arylsulfinyl group having 6 to 30 carbon atoms, e.g. a methylsulfinyl group, an ethylsulfinyl group, a phenylsulfinyl group, or a p-methylphenylsulfinyl group); an alkyl- or aryl-sulfonyl group (preferably a substituted or unsubstituted alkylsulfonyl group having 1 to 30 carbon atoms, or a substituted or unsubstituted arylsulfonyl group having 6 to 30 carbon atoms, e.g. a methylsulfonyl group, an ethylsulfonyl group, a phenylsulfonyl group, or a p-methylphenylsulfonyl group); an acyl group (preferably a formyl group, a substituted or unsubstituted alkylcarbonyl group having 2 to 30 carbon atoms, a substituted or unsubstituted arylcarbonyl group having 7 to 30 carbon atoms, or a substituted or unsubstituted heterocyclic carbonyl group having 4 to 30 carbon atoms and being bonded to said carbonyl group through a carbon atom, e.g. an acetyl group, a pivaloyl group, a 2-chloroacetyl group, a stearoyl group, a benzoyl group, a p-n-octyloxyphenylcarbonyl group, a 2-pyridylcarbonyl group, or a 2-furylcarbonyl group); an aryloxycarbonyl group (preferably a substituted or unsubstituted aryloxycarbonyl group having 7 to 30 carbon atoms, e.g. a phenoxycarbonyl group, an o-chlorophenoxycarbonyl group, an m-nitrophenoxycarbonyl group, or a p-t-butylphenoxycarbonyl group); an alkoxycarbonyl group (preferably a substituted or unsubstituted alkoxycarbonyl group having 2 to 30 carbon atoms, e.g. a methoxycarbonyl group, an ethoxycarbonyl group, a t-butoxycarbonyl group, or an n-octadecyloxycarbonyl group); a carbamoyl group (preferably a substituted or unsubstituted carbamoyl group having 1 to 30 carbon atoms, e.g. a carbamoyl group, an N-methylcarbamoyl group, an N,N-dimethylcarbamoyl group, an N,N-di-n-octylcarbamoyl group, or an N-(methylsulfonyl)carbamoyl group); an aryl- or heterocyclic-azo group (preferably a substituted or unsubstituted aryl azo group having 6 to 30 carbon atoms, or a substituted or unsubstituted heterocyclic azo group having 3 to 30 carbon atoms, e.g. a phenylazo group, a p-chlorophenylazo group, or a 5-ethylthio-1,3,4-thiadiazol-2-ylazo group); an imido group (preferably an N-succinimido group or an N-phthalimido group); a phosphino group (preferably a substituted or unsubstituted phosphino group having 2 to 30 carbon atoms, e.g. a dimethylphosphino group, a diphenylphosphino group, or a methylphenoxyphosphino group); a phosphinyl group (preferably a substituted or unsubstituted phosphinyl group having 2 to 30 carbon atoms, e.g. a phosphinyl group, a dioctyloxyphosphinyl group, or a diethoxyphosphinyl group); a phosphinyloxy group (preferably a substituted or unsubstituted phosphinyloxy group having 2 to 30 carbon atoms, e.g. a diphenoxyphosphinyloxy group or a dioctyloxyphosphinyloxy group); a phosphinylamino group (preferably a substituted or unsubstituted phosphinylamino group having 2 to 30 carbon atoms, e.g. a dimethoxyphosphinylamino group or a dimethylaminophosphinylamino group); a silyl group (preferably a substituted or unsubstituted silyl group having 3 to 30 carbon atoms, e.g. a trimethylsilyl group, a t-butyidimethylsilyl group, or a phenyldimethylsilyl group).

Among the above functional groups, those having a hydrogen atom may further be substituted with any of the above groups at the position from which the hydrogen atom is removed. Examples of such a functional group include an alkylcarbonylaminosulfonyl group, an arylcarbonylaminosulfonyl group, an alkylsulfonylaminocarbonyl group, and an arylsulfonylaminocarbonyl group. Specific examples of these groups include methylsulfonylaminocarbonyl, p-methylphenylsulfonylaminocarbonyl, acetylaminosulfonyl, and benzoylaminosulfonyl.

Examples of the substituent on the aryl moiety of the substituted aralkyl group include substituents of the following substituted aryl groups.

The aromatic group in this specification means an aryl group or a substituted aryl group. Further, these aromatic groups may be condensed with aliphatic rings, other aromatic rings or hetero rings. The number of carbon atoms of the aromatic group is preferably 6 to 40, more preferably 6 to 30, and still more preferably 6 to 20. Among these groups, the aryl group is preferably phenyl or naphthyl, and particularly preferably phenyl.

The aryl moiety of the substituted aryl group is the same as the above aryl group. Examples of the substituent of the substituted aryl group include those given above as the substituents of the substituted alkyl group, the substituted alkenyl group, the substituted alkynyl group, and the alkyl moiety of the substituted aralkyl group.

In the present specification, a heterocyclic group preferably contains a 5- or 6-membered saturated or unsaturated heterocycle. Such a heterocycle may be condensed with an aliphatic ring, an aromatic ring, or another heterocycle. Examples of the heteroatom in the heterocycle include B, N, O, S, Se, and Te. As a heteroatom, N, O, and S are preferable. The heterocycle preferably has a free monovalent carbon atom (the heterocyclic group binds at the carbon atom). The heterocyclic group has preferably 1 to 40 carbon atoms, more preferably 1 to 30 carbon atoms, and further more preferably 1 to 20 carbon atoms. Examples of the saturated heterocycle include a pyrrolidine ring, a morpholine ring, 2-bora-1,3-dioxolane ring, and 1,3-thiazolidine ring. Examples of the unsaturated heterocycle include an imidazole ring, a thiazole ring, a benzothiazole ring, a benzoxazole ring, a benzotriazole ring, a benzoselenazole ring, a pyridine ring, a pyrimidine ring, and a quinoline ring. The heterocyclic group may have a substituent or substituents. Examples of the substituent include the substituents listed above as the substituents for the substituted alkyl group, the substituted alkenyl group, the substituted alkynyl group, and the alkyl moiety of the substituted aralkyl group.

The compounds represented by formulas (I) to (III) are explained below.

In formulas (I) to (III), Ar³¹ is a divalent aromatic group or a divalent heterocyclic group. Each of these groups is a group where a single hydrogen atom of the corresponding aromatic or heterocyclic group as described above is replaced with a bond. Ar³¹ is preferably a 1,4-phenylene or a pyridine-2,5-diyl (including two types: a structure having the substituent —NR³¹R³² at the 5-position of the pyridine ring and a structure having the substituent —NR³¹R³² at the 2-position of the pyridine ring), and most preferably a 1,4-phenylene. Examples of the substituent on Ar³¹ include the substituents listed above for the substituted alkyl group, the substituted alkenyl group, the substituted alkynyl group, and the alkyl moiety of the substituted aralkyl group. Preferable examples of the substituent include a halogen atom, an alkyl group, an alkenyl group, an alkynyl group, an aryl group, a cyano group, a hydroxyl group, a nitro group, a carboxyl group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkylsulfonylamino group, an arylsulfonylamino group, a mercapto group, an alkyl thio group, an aryl thio group, a heterocyclic thio group, a sulfamoyl group, a sulfo group, an alkyl sulfinyl group, an aryl sulfinyl group, an alkyl sulfonyl group, an aryl sulfonyl group, an acyl group, an aryloxycarbonyl group, an alkoxycarbonyl group, a carbamoyl group, an imido group, a phosphino group, a phosphinyl group, a phosphinyloxy group, a phosphinylamino group, and a silyl group. More preferred examples include a halogen atom, an alkyl group, an aryl group, a cyano group, a nitro group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkylsulfonylamino group, an arylsulfonylamino group, an alkyl thio group, an aryl thio group, a heterocyclic thio group, a sulfamoyl group, an alkyl sulfinyl group, an aryl sulfinyl group, an alkyl sulfonyl group, an aryl sulfonyl group, an acyl group, an aryloxycarbonyl group, an alkoxycarbonyl group, a carbamoyl group, an imido group, and a silyl group. Further preferred examples include a halogen atom, an alkyl group, a cyano group, an alkoxy group, an amino group, an acylamino group, an aminocarbonylamino group, an alkoxycarbonylamino group, an aryloxycarbonylamino group, a sulfamoylamino group, an alkylsulfonylamino group, an arylsulfonylamino group, a sulfamoyl group, an alkyl sulfinyl group, an aryl sulfinyl group, an alkyl sulfonyl group, and an aryl sulfonyl group. Furthermore preferred examples include a halogen atom, an alkyl group, an alkoxy group, an acylamino group, an aminocarbonylamino group, an alkoxycarbonylamino group, an alkylsulfonylamino group. Furthermore preferred examples include a halogen atom, an alkyl group having 1 to 3 carbon atoms, an alkoxy group having 1 to 3 carbon atoms, an acylamino group having 2 to 3 carbon atoms, an aminocarbonylamino group having 1 to 4 carbon atoms, an alkylsulfonylamino group having 1 to 3 carbon atoms. Particularly preferred examples include an alkyl group having 1 to 3 carbon atoms, an alkoxy group having 1 to 3 carbon atoms, an acylamino group having 2 to 3 carbon atoms, an aminocarbonylamino group having 1 to 3 carbon atoms, an alkylsulfonylamino group having 1 to 3 carbon atoms.

When L²¹ is —N(R²⁶)—, R²⁶ is preferably a hydrogen atom, an alkyl group having 1 to 6 carbon atoms, or a phenyl group having 6 to 8 carbon atoms; more preferably a hydrogen atom or an alkyl group having 1 to 3 carbon atoms, and most preferably a hydrogen atom or a methyl group. L²¹ is preferably a single bond or —O—, and most preferably a single bond.

Z²¹ is preferably —O— or —N(R²⁷)—, wherein R²⁷ is preferably a hydrogen atom, an alkyl group, an alkenyl group, an alkynyl group, an aryl group, an amino group, an imino group, an acylamino group, an aminocarbonylamino group, an alkoxycarbonylamino group, an aryloxycarbonylamino group, a sulfamoylamino group, an alkyl- or aryl-sulfonylamino group, or an acyl group. Z²¹ is more preferably —O— or —N(R²⁷)—, wherein R²⁷ is an alkyl group, an alkenyl group, an amino group, an imino group, an acylamino group, an alkyl- or aryl-sulfonylamino group, or an acyl group. Z²¹ is still more preferably —O— or —N(R²⁷)—, wherein R²⁷ is an alkenyl group, an amino group, an imino group, or an acylamino group. More preferred is a case where Z²¹ is —O— or —N(R²⁷)—, Z²¹ and R²⁴ in formula (II) form a ring as shown in formula (II-1) or (II-2). Still more preferred is a case where Z²¹ is —N(R²⁷)— and Z²¹ and R²⁴ in formula (II) form a ring as shown in formula (II-1). Most preferred is a case where Z²¹ is —N(R²⁷)—, Z²¹ and R²⁴ in formula (II) form a ring as shown in formula (II-1), and A¹ is a nitrogen atom.

Preferred examples of R²¹, R²², R²³, and R²⁴ include a hydrogen atom, a halogen atom, an alkyl group, an alkenyl group, an alkynyl group, an aryl group, a cyano group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group, an acylamino group, an imino group, an aminocarbonylamino group, an alkoxycarbonylamino group, an aryloxycarbonylamino group, a sulfamoylamino group, an alkylsulfonylamino group, an arylsulfonylamino group, an alkyl thio group, an aryl thio group, a heterocyclic thio group, a sulfamoyl group, a sulfo group, an alkyl sulfinyl group, an aryl sulfinyl group, an alkyl sulfonyl group, an aryl sulfonyl group, an acyl group, an aryloxycarbonyl group, an alkoxycarbonyl group, a carbamoyl group, an imido group, a phosphino group, a phosphinyl group, a phosphinyloxy group, and a phosphinylamino group. More preferred examples include a hydrogen atom, a halogen atom, an alkyl group, an alkenyl group, an aryl group, a cyano group, an alkoxy group, an aryloxy group, an acyloxy group, an amino group, an imino group, an acylamino group, an alkyl thio group, an aryl thio group, a sulfamoyl group, a sulfo group, an acyl group, an aryloxycarbonyl group, and an alkoxycarbonyl group. More preferred examples include a hydrogen atom, a halogen atom, an alkyl group, an alkenyl group, an aryl group, a cyano group, an alkoxy group, an aryloxy group, an amino group, an imino group, an acylamino group, an alkyl thio group, an aryl thio group, a sulfo group, an acyl group, an aryloxycarbonyl group, and an alkoxycarbonyl group. The formation of formula (I-1) or (I-2) is preferred, and the formation of formula (I-1) with A¹ being a nitrogen atom is more preferred.

R²⁵ is preferably a hydrogen atom, an alkyl group having 1 to 20 carbon atoms, an alkenyl group having 1 to 20 carbon atoms, an aryl group having 6 to 20 carbon atoms, or a heterocyclic group having 2 to 20 carbon atoms; more preferably a hydrogen atom, an alkyl group having 1 to 10 carbon atoms, an aryl group having 6 to 10 carbon atoms, or a heterocyclic group having 2 to 10 carbon atoms; still more preferably an alkyl group having 1 to 6 carbon atoms, a phenyl group having 6 to 8 carbon atoms, or a heterocyclic group having 2 to 6 carbon atoms; yet more preferably methyl, ethyl, isopropyl, propyl, butyl, isobutyl, phenyl, 2-pyridyl, 3-pyridyl, 4-pyridyl, or 3-triazolyl; even more preferably methyl, ethyl, phenyl, or 3-pyridyl; still yet more preferably methyl or phenyl; most preferably methyl.

R³¹ and R³² are each preferably a hydrogen atom, an alkyl group having 1 to 15 carbon atoms, an alkenyl group having 2 to 15 carbon atoms, or an aryl group having 6 to 15 carbon atoms; more preferably an alkyl group having 1 to 10 carbon atoms, an alkenyl group having 2 to 10 carbon atoms, or an aryl group having 6 to 10 carbon atoms; still more preferably an alkyl group having 1 to 10 carbon atoms. n₂₁ is preferably 0.

In formula (I), the moiety other than ═N—Ar³¹—N(R³¹)(R³²) is derived from a coupler component that is necessary to complete an azomethine dye of formula (I). Examples of the coupler compound include phenols; naphthols; active-hydrogen-containing heterocyclic compounds, such as 5-pyrazolones, 1H-pyrazolo[1,5-a]benzimidazoles, 1H-pyrazolo[5,1-c]-1,2,4-triazoles, 1H-pyrazolo[2,3-b]-1,2,4-triazoles, 1H-1,2,4-triazoles, 2,4-diphenylimidazoles, pyrazolopyrimidine-7-ones, pyrazolopyrimidine-5-ones, pyrazoloquinazolones, pyrazolo[5,1-c][1,2,4]triazine-7-ones, imidazopyrazoles, and pyrazolopyrimidinediones; and active methylene compounds, such as acylacetonitriles, acylacetanilides, diacylmethanes, malondianilides, and β,γ-unsaturated nitriles.

Next, the compounds represented by formula (I-1), (I-2), (II-1), (II-2), or (III-1) will be explained.

In formula (I-1), (I-2), (II-1), (II-2), or (III-1), L²¹¹ and L²²¹ each preferably have the same meaning as L²¹ and also have the same preferred range.

With respect to A¹ and A², one represents a nitrogen atom, while the other represents —C(R²¹⁴)═, wherein R²¹⁴ represents a hydrogen atom or a substituent. Preferred examples of R²¹⁴ include a hydrogen atom, a halogen atom, an alkyl group, an alkenyl group, an alkynyl group, an aryl group, a cyano group, a hydroxyl group, a nitro group, a carboxyl group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group, an imino group, an acylamino group, an aminocarbonylamino group, an alkoxycarbonylamino group, an aryloxycarbonylamino group, a sulfamoylamino group, an alkylsulfonylamino group, an arylsulfonylamino group, a mercapto group, an alkyl thio group, an aryl thio group, a heterocyclic thio group, a sulfamoyl group, a sulfo group, an alkyl sulfinyl group, an aryl sulfinyl group, an alkyl sulfonyl group, an aryl sulfonyl group, an acyl group, an aryloxycarbonyl group, an alkoxycarbonyl group, a carbamoyl group, an imido group, a phosphino group, a phosphinyl group, a phosphinyloxy group, a phosphinylamino group, and a silyl group. More preferred examples of R²¹⁴ include a hydrogen atom, an alkyl group, an alkenyl group, an alkynyl group, an aryl group, a cyano group, a nitro group, an alkoxy group, an aryloxy group, a silyloxy group, a heterocyclic oxy group, an acyloxy group, a carbamoyloxy group, an amino group, an acylamino group, an alkyl thio group, an aryl thio group, a heterocyclic thio group, a sulfamoyl group, a sulfo group, an alkyl sulfinyl group, an aryl sulfinyl group, an alkyl sulfonyl group, an aryl sulfonyl group, an acyl group, an aryloxycarbonyl group, an alkoxycarbonyl group, a carbamoyl group, an imido group, and a silyl group. Furthermore preferred examples of R²¹⁴ include a hydrogen atom, an alkyl group, an alkenyl group, an alkynyl group, an aryl group, a cyano group, an alkoxy group, an aryloxy group, a silyloxy group, a heterocyclic oxy group, an acylamino group, an alkyl thio group, an aryl thio group, a heterocyclic thio group, and an aryloxycarbonyl group. Furthermore preferred examples of R²¹⁴ include a hydrogen atom, an alkyl group having 1 to 60 carbon atoms, an alkenyl group having 1 to 60 carbon atoms, and an aryl group having 6 to 60 carbon atoms. Furthermore preferred examples of R²¹⁴ include a hydrogen atom, an alkyl group having 1 to 60 carbon atoms, and an aryl group having 6 to 60 carbon atoms.

In a preferred combination of A¹ and A², A¹ is a nitrogen atom.

X and Y are each a nitrogen atom or ═C(R³¹⁵)—, wherein R³¹⁵ is a hydrogen atom or a substituent. Preferred is a case where one of X and Y is a nitrogen atom, or both of X and Y are ═C(R³¹⁵)—. More preferred is a case where one of X and Y is a nitrogen atom or both of X and Y are ═C(R³¹⁵)—, wherein R³¹⁵ for X is a hydrogen atom, an alkyl group, an alkoxy group, an acylamino group, an aminocarbonylamino group, or an alkylsulfonylamino group, and R³¹⁵ for Y is a hydrogen atom or an alkyl group. Still more preferred is a case where both of X and Y are ═C(R³¹⁵)—, wherein R³¹⁵ for X is a hydrogen atom, an alkyl group, an alkoxy group, an acylamino group, an aminocarbonylamino group, or an alkylsulfonylamino group, and R³¹⁵ for Y is a hydrogen atom or an alkyl group that forms a ring together with R³¹⁴. Yet more preferred is a case where both of X and Y are ═C(R³¹⁵)—, wherein R³¹⁵ for X is a hydrogen atom, an alkyl group having 1 to 3 carbon atoms, an acylamino group having 2 to 3 carbon atoms, an aminocarbonylamino group having 1 to 3 carbon atoms, or an alkylsulfonylamino group having 1to 3 carbon atoms, and R³¹⁵ for Y is a hydrogen atom or an alkyl group that forms a ring together with R³¹⁴. Even more preferred is a case where both of X and Y are ═C(R³¹⁵)—, wherein R³¹⁵ for X is a hydrogen atom, an alkyl group having 1 to 3 carbon atoms, an acylamino group having 2 to 3 carbon atoms, an aminocarbonylamino group having 1 to 3 carbon atoms, or an alkylsulfonylamino group having 1 to 3 carbon atoms, and R³¹⁵ for Y is a hydrogen atom.

R²¹¹ preferably has the same meaning as R²¹ and also has the same preferred range. R²¹¹ is preferably a hydrogen atom, a halogen atom, an alkyl group, an aryl group, an alkoxy group, an aryloxy group, an alkylthio group, or an arylthio group; more preferably a hydrogen atom, a chlorine atom, an alkyl group having 1 to 10 carbon atoms, an aryl group having 6 to 10 carbon atoms, an alkoxy group having 1 to 10 carbon atoms, or an aryloxy group having 6 to 10 carbon atoms; still more preferably a hydrogen atom, an alkyl group having 1 to 5 carbon atoms, an aryl group having 6 to 8 carbon atoms, or an aryloxy group having 6 to 10 carbon atoms; yet more preferably an alkyl group having 1 to 4 carbon atoms or a phenyl group having 6 to 8 carbon atoms.

R²¹² and R²²⁴ each preferably have the same meaning as R²⁵ and also have the same preferred range. R²²¹ and R²²² each are an electron attractive group of which the Hammett's substituent constant σₚ value (hereinafter, simply referred to as σₚ value) is 0.20 or more and 1.0 or less. Preferably R²²¹ and R²²² each are an electron attractive group of which the σₚ value is 0.30 or more and 0.80 or less. The Hammett rule and σₚ values are described in detail in, for example, “Lange's Handbook of Chemistry”, 12th edition, (1979), edited by J. A. Dean (McGraw-Hill); “Kagaku No Ryoiki” (Region of Chemistry), extra edition, No. 122, pp. 96-103, (1979) (Nankodo); and “Chemical Reviews”, Vol. 91, pp. 165-195, (1991). In the present invention, R²²¹ and R²²² are defined in terms of the Hammett substituent constant σₚ value, but this does not mean that the substituent is limited to those having a value known in the literatures, which can be found in the above literatures; it is needless to say that even if the value is unknown in any literature, substituents which can have a value in the range if measured according to the Hammett rule are also included in the present invention. Furthermore preferred examples of R²²¹ and R²²² include an acyl group, an acyloxy group, a carbamoyl group, an alkoxycarbonyl group, an aryloxycarbonyl group, a cyano group, a nitro group, an alkylsulfinyl, an arylsulfinyl group, an alkylsulfonyl group, an arylsulfonyl group, a sulfamoyl group, a halogenated alkyl group, a halogenated alkoxy group, a halogenated alkylthio group, a halogenated aryloxy group, an aryl group substituted by at least two electron-attracting groups whose σₚ values are 0.20 or more, and a heterocyclic group. Still more preferred examples include an alkoxycarbonyl group, a nitro group, a cyano group, an arylsulfonyl group, a carbamoyl group, and a halogenated alkyl group. Particularly preferred is a case where R²²¹ is a cyano group and R²²² is an alkoxycarbononyl group.

Preferred example of R²²³ include an alkyl group, an alkenyl group, an alkynyl group, an aryl group, a cyano group, an alkoxy group, an aryloxy group, a silyloxy group, a heterocyclic oxy group, an acyloxy group, a carbamoyloxy group, an alkoxycarbonyloxy group, an aryloxycarbonyloxy group, an acylamino group, an aminocarbonylamino group, an alkoxycarbonylamino group, an aryloxycarbonylamino group, a sulfamoylamino group, an alkylsulfonylamino group, an arylsulfonylamino group, an alkyl thio group, an aryl thio group, a heterocyclic thio group, a sulfamoyl group, an alkyl sulfinyl group, an aryl sulfinyl group, an alkyl sulfonyl group, an aryl sulfonyl group, an acyl group, an aryloxycarbonyl group, an alkoxycarbonyl group, a carbamoyl group, an imido group, and a silyl group. More preferred example of R²²³ include an alkyl group, an alkenyl group, an alkynyl group, an aryl group, an alkoxy group, an aryloxy group, and a silyl group. Further preferred examples of R²²³ include an alkyl group, an alkenyl group, and an aryl group. Furthermore preferred examples of R²²³ include an alkyl group and an aryl group.

When X is a nitrogen atom, R³¹¹ is preferably a hydrogen atom, an alkyl group, an alkoxy group, an acylamino group, an aminocarbonylamino group, or an alkylsulfonylamino group; more preferably a hydrogen atom, an alkyl group having 1 to 3 carbon atoms, an acylamino group having 2 to 3 carbon atoms, an aminocarbonylamino group having 1 to 3 carbon atoms, or an alkylsulfonylamino group having 1 to 3 carbon atoms. When X is a carbon atom, R³¹¹ is preferably a hydrogen atom. R³¹² is preferably a hydrogen atom or an alkyl group that forms a ring together with R³¹⁴, more preferably a hydrogen atom.

R³¹³ and R³¹⁴ are each preferably an alkyl group, an alkenyl group, or an aryl group; more preferably an alkyl group having 1 to 20 carbon atoms, an alkenyl group having 2 to 20 carbon atoms, or an aryl group having 6 to 20 carbon atoms; still more preferably an alkyl group having 1 to 10 carbon atoms or an aryl group having 6 to 10 carbon atoms; yet more preferably an alkyl group having 1 to 10 carbon atoms. Also preferably, R³¹³ and R³¹⁴ each may form a ring together with any other substituent.

Next, specific examples of the compounds represented by formula (I), (II), or (III) according to the present invention are shown below. However, the present invention should not be construed as being limited to these compounds.

Specific examples of the compound represented by formula (I) are shown below.

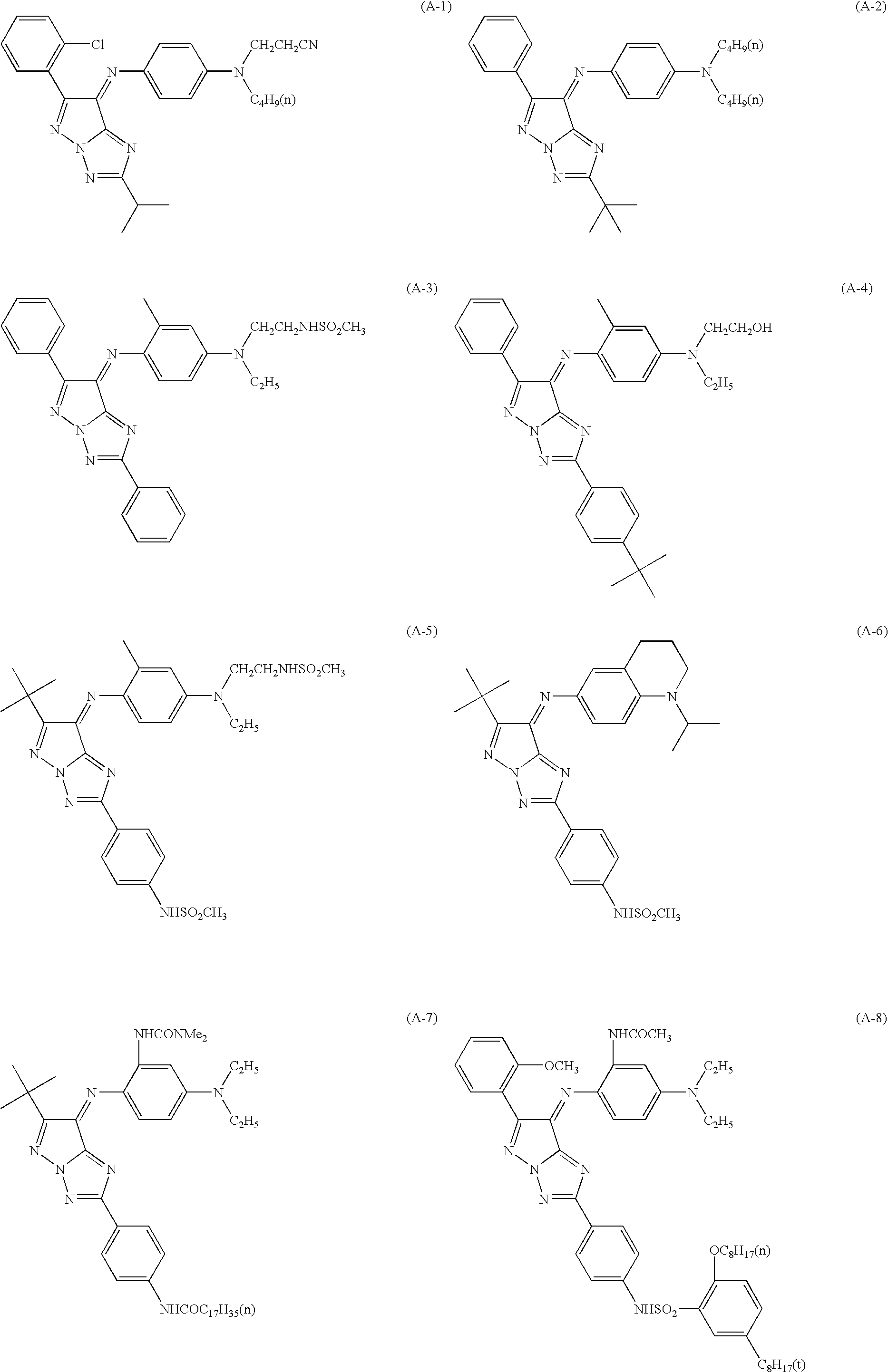

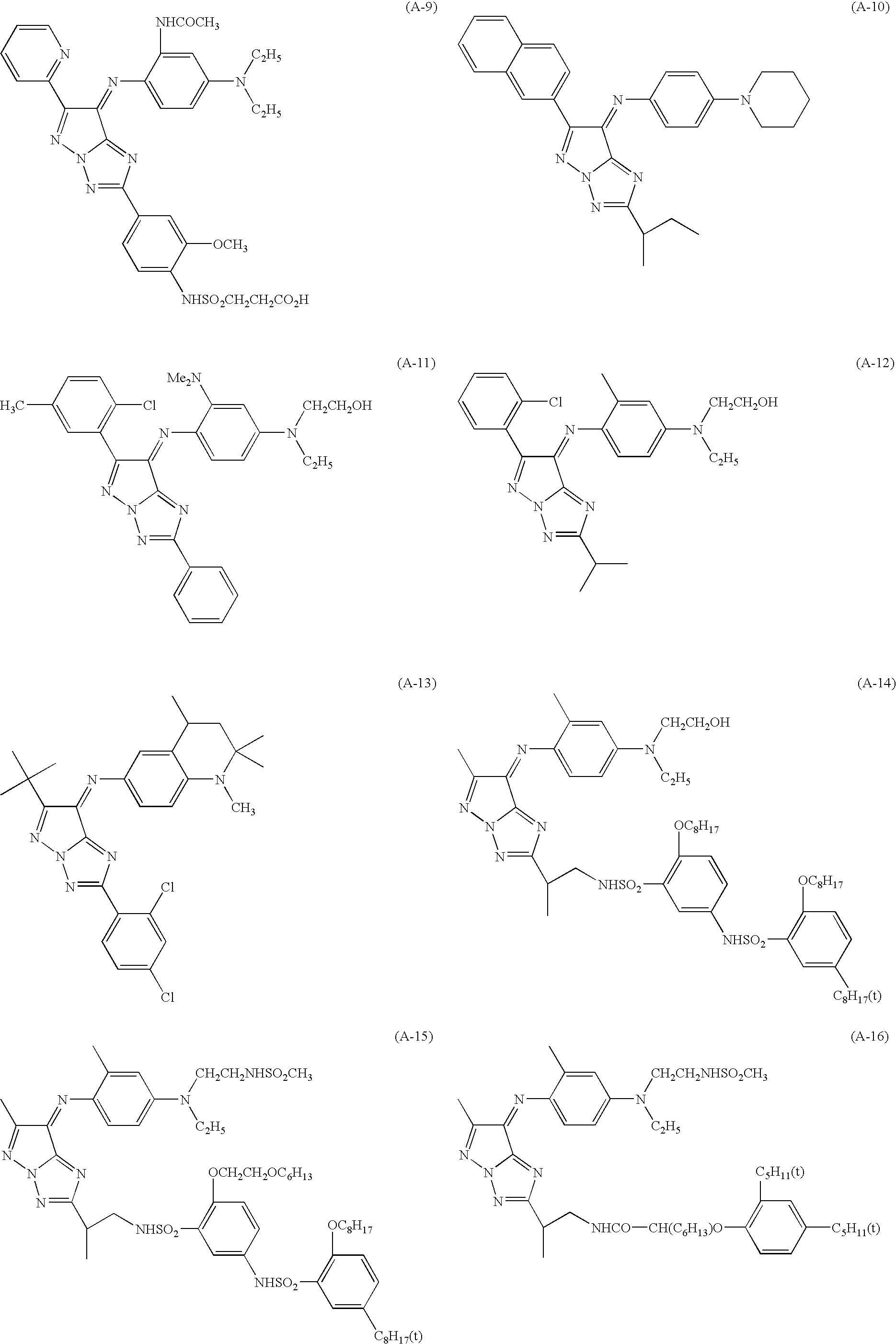

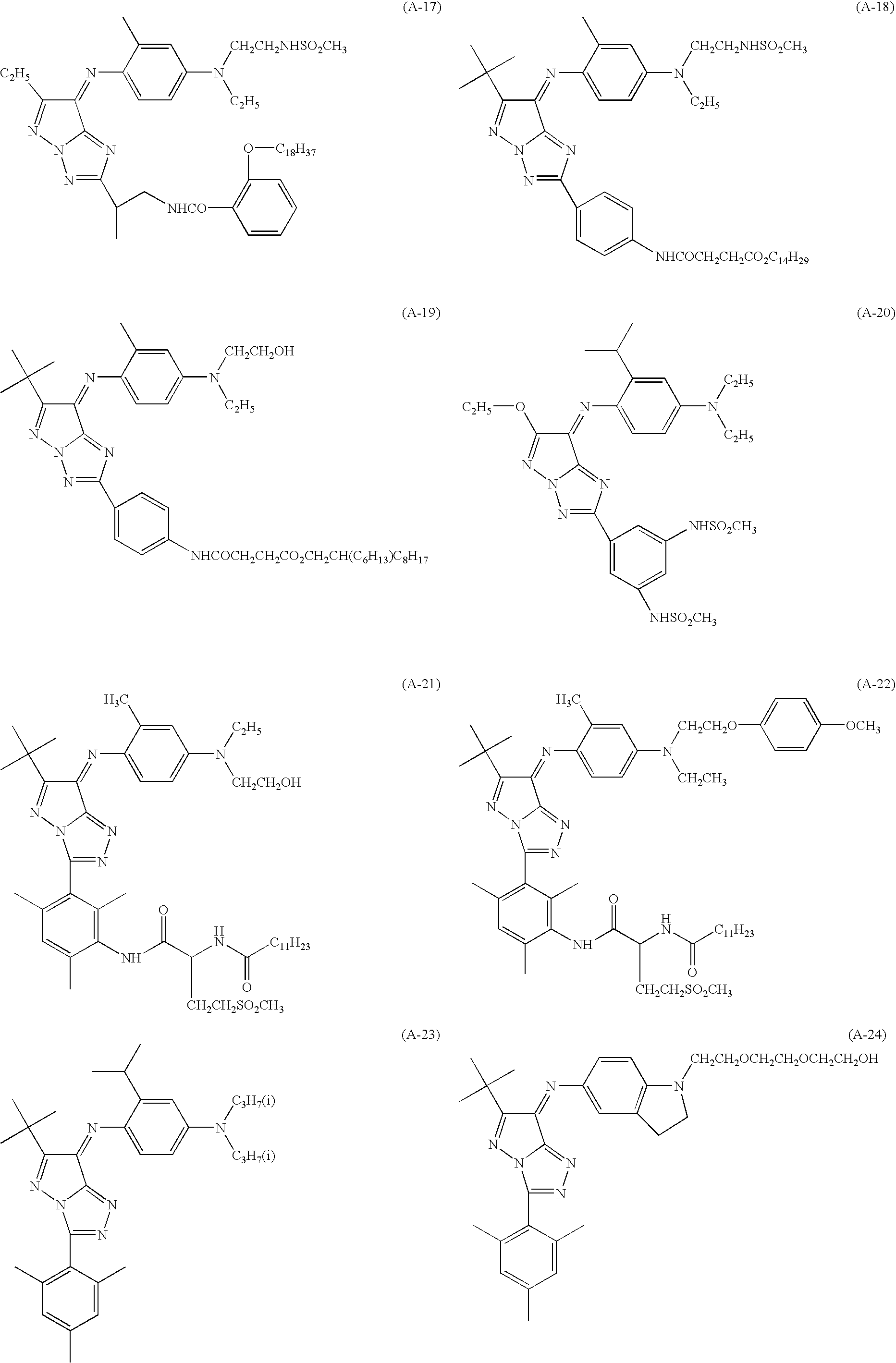

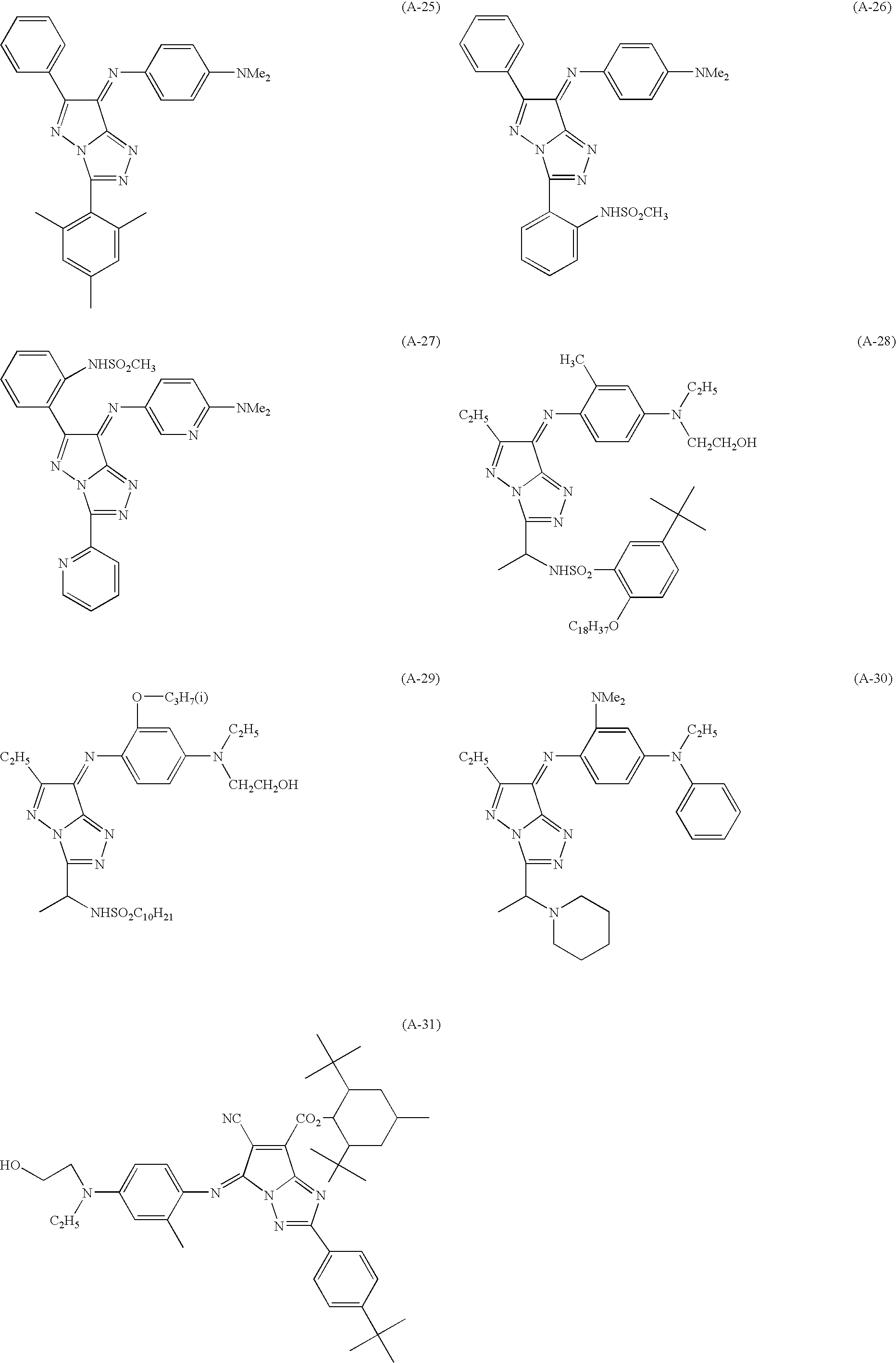

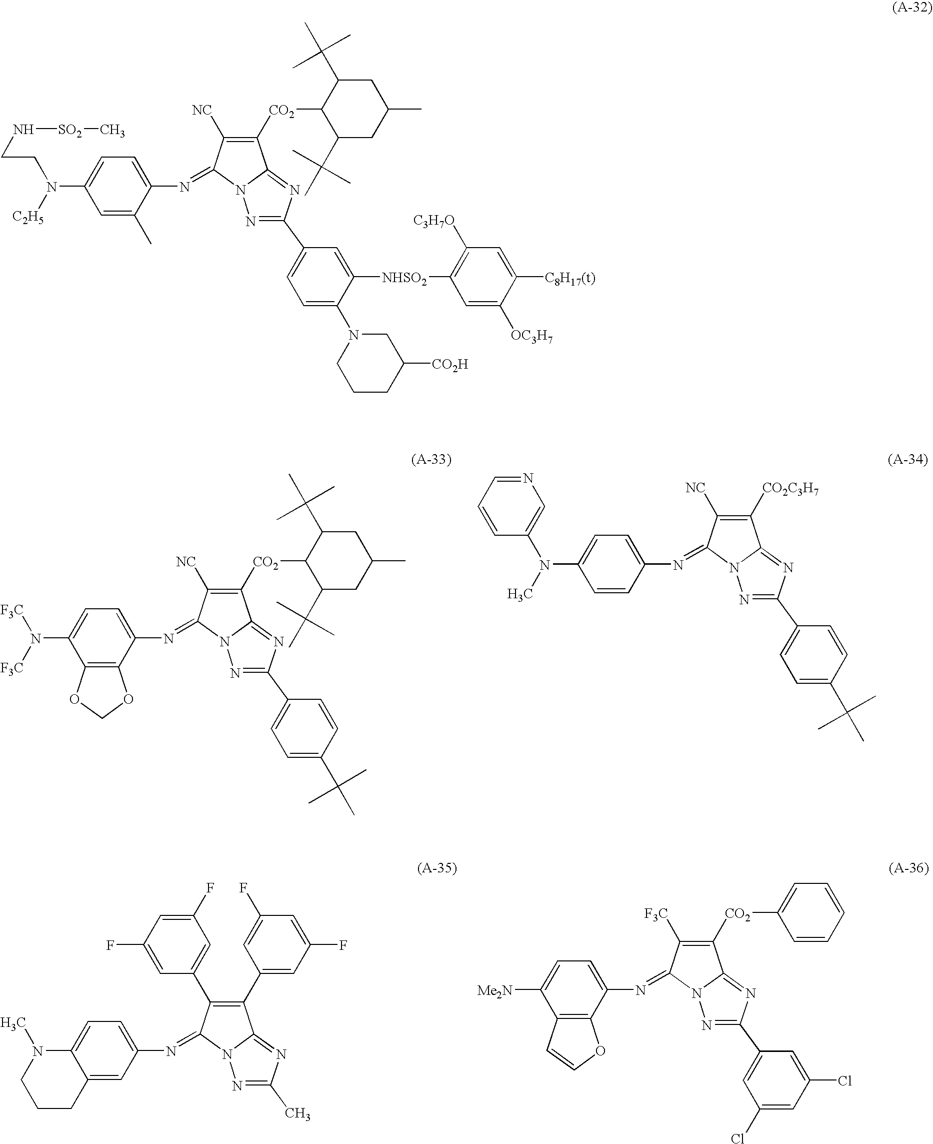

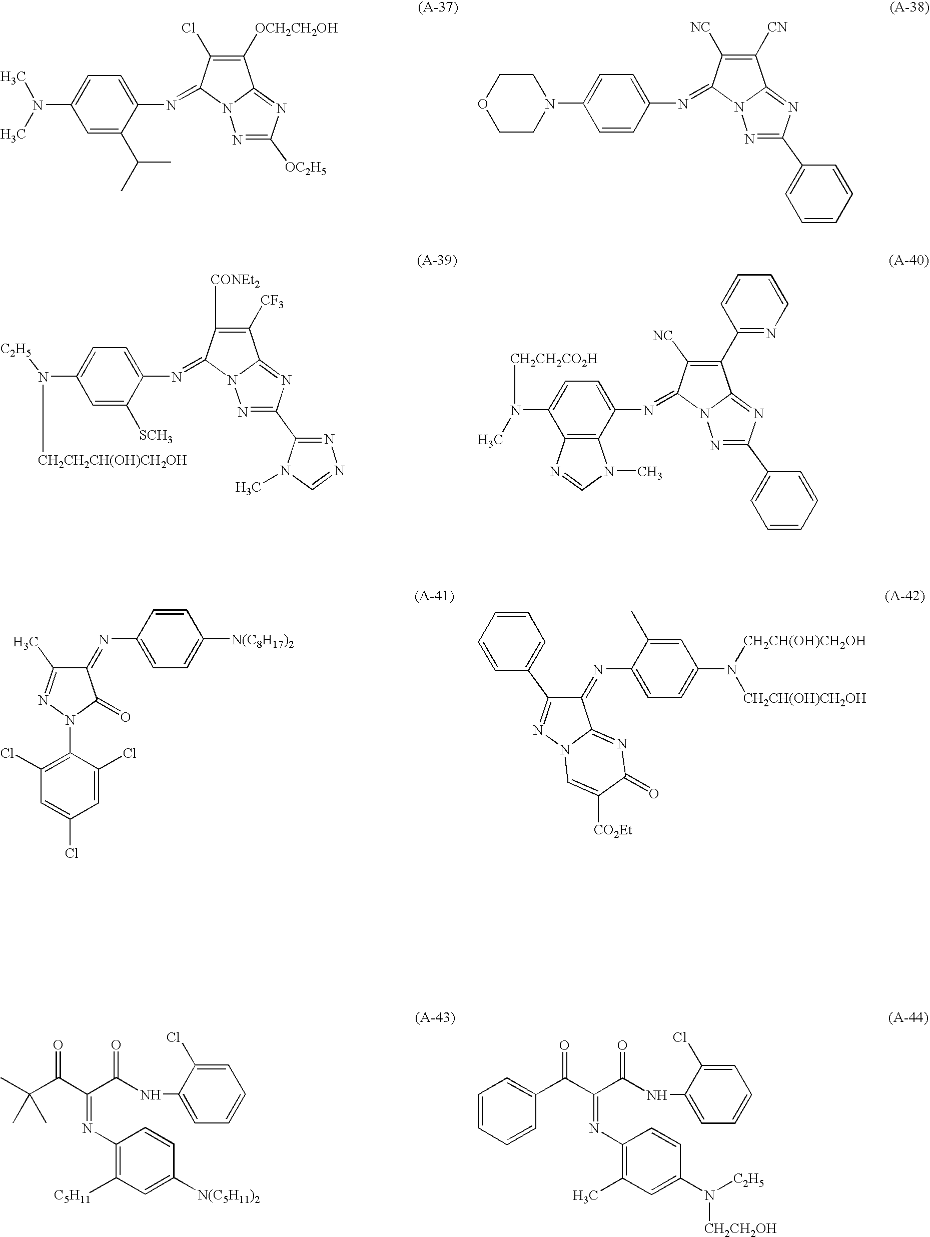

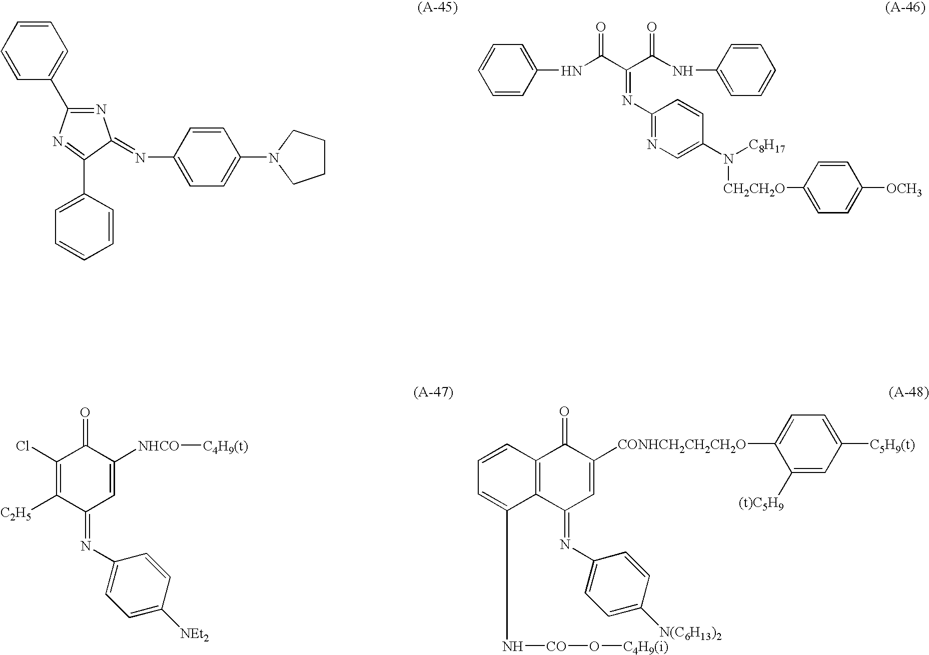

Specific examples of the compound represented by formula (II) are shown below.

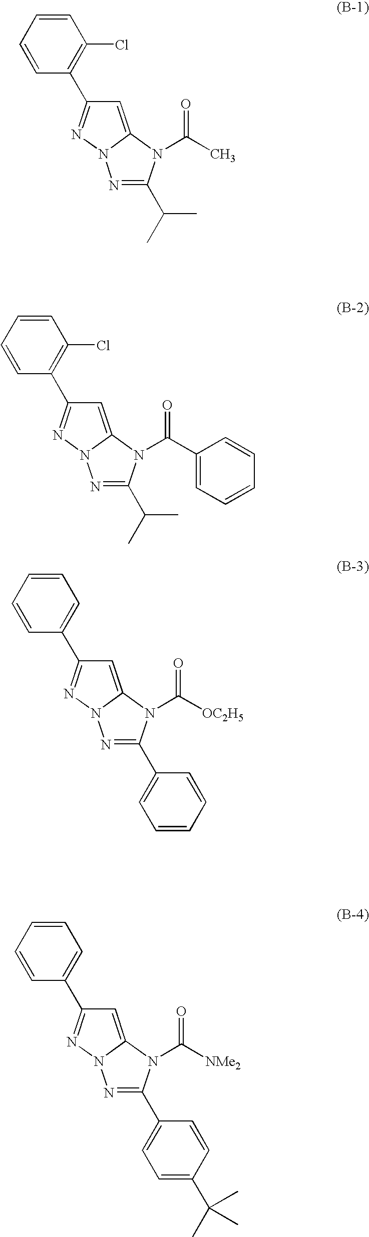

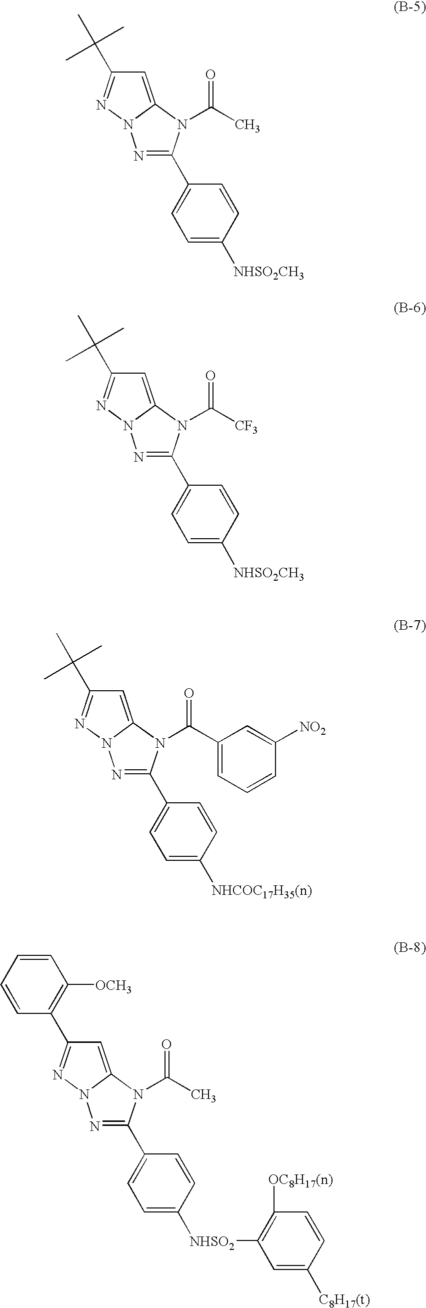

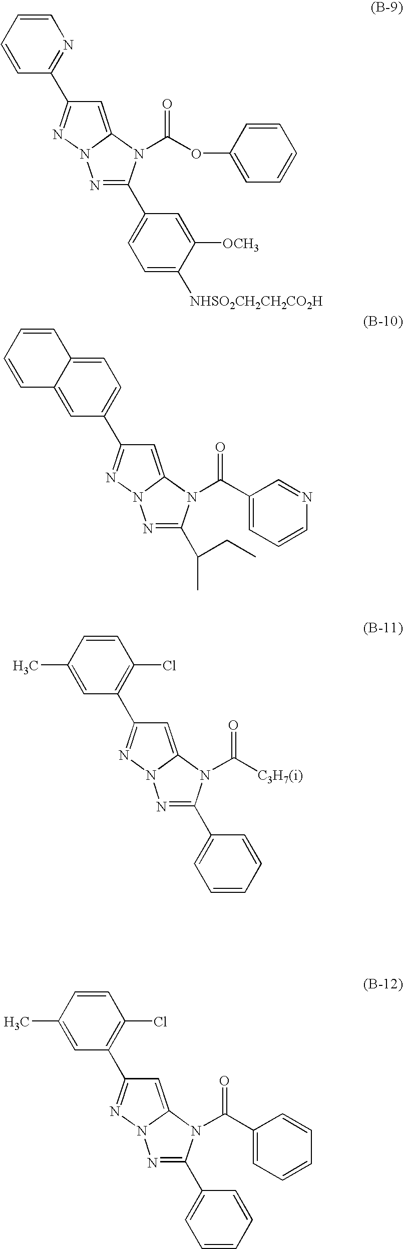

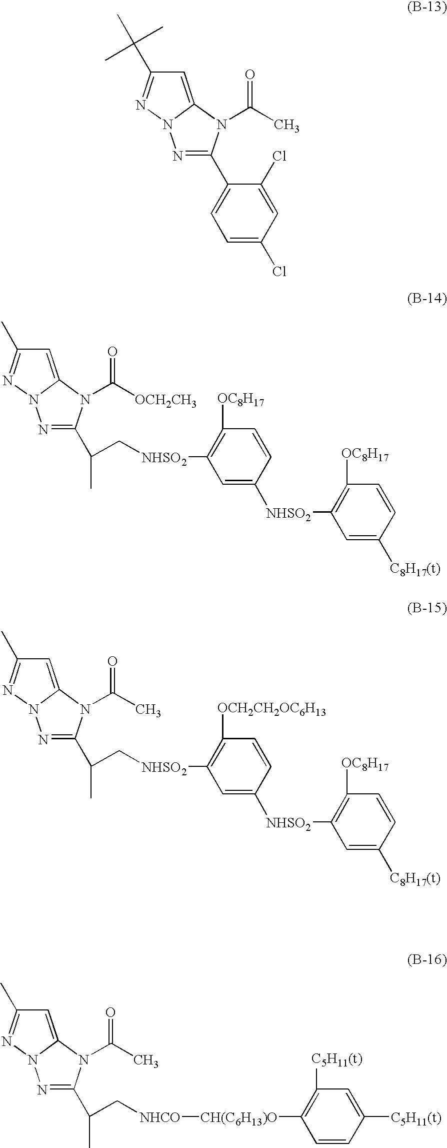

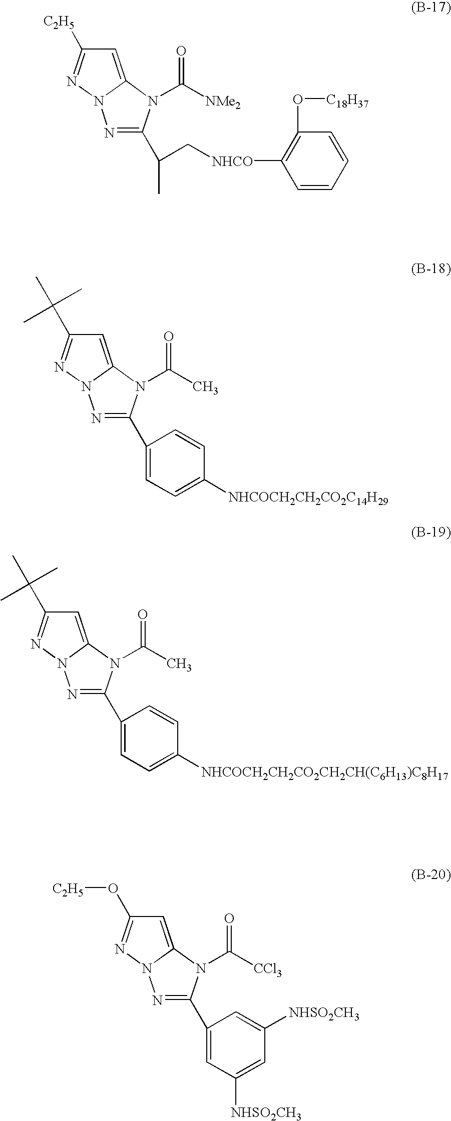

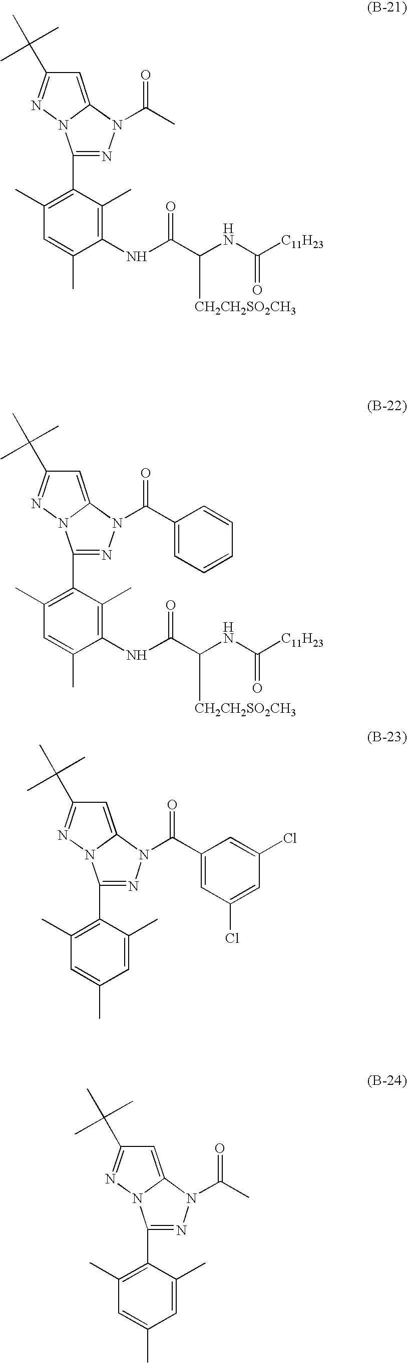

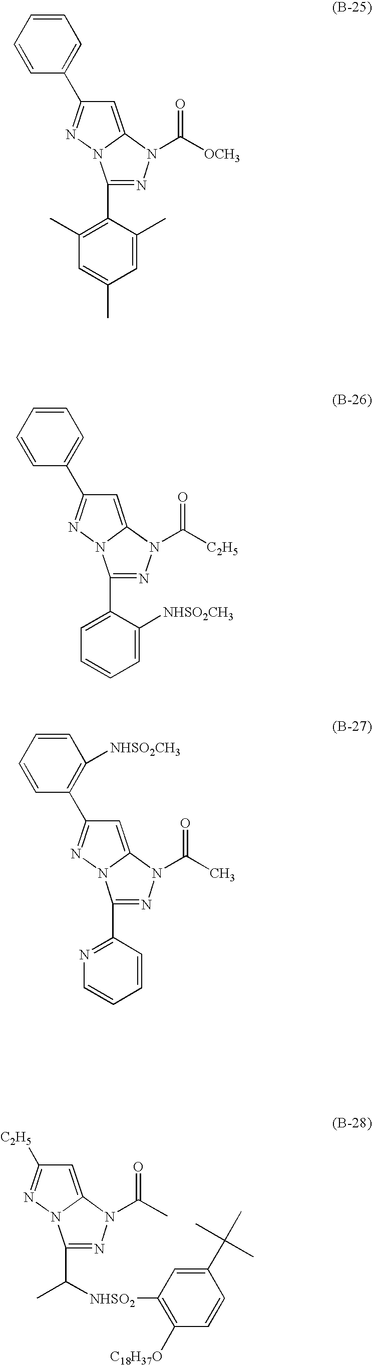

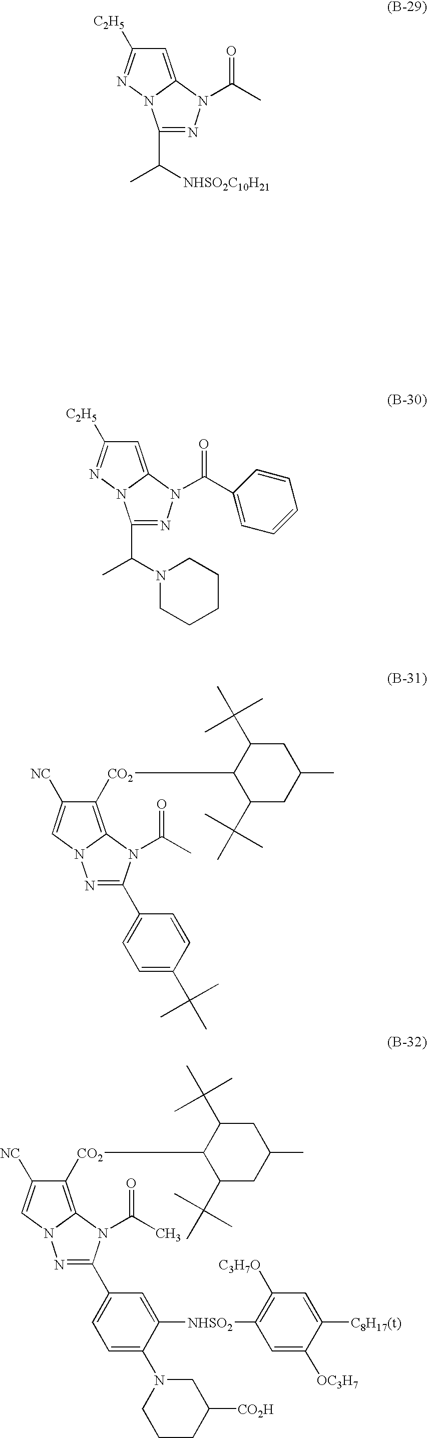

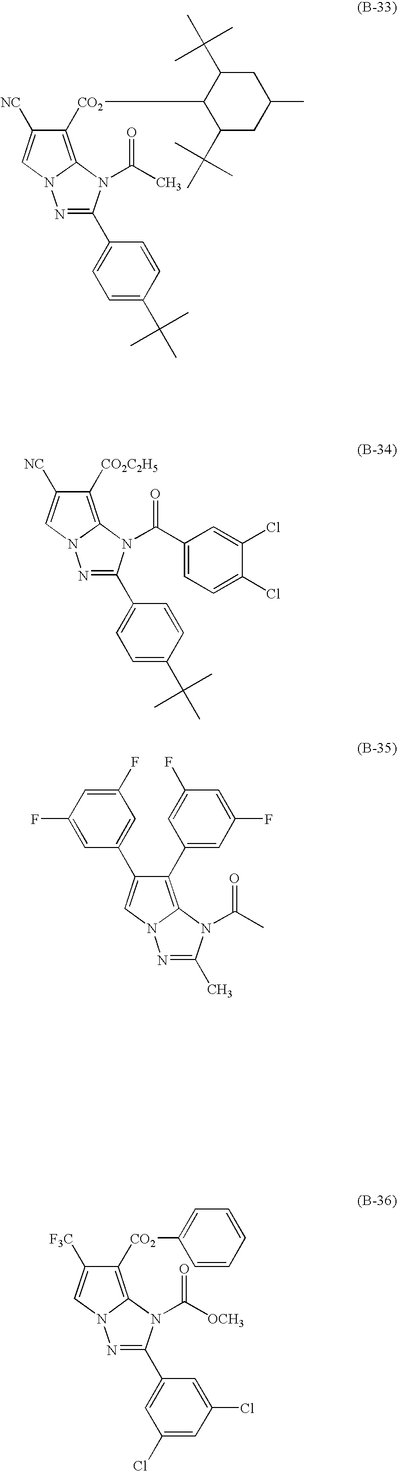

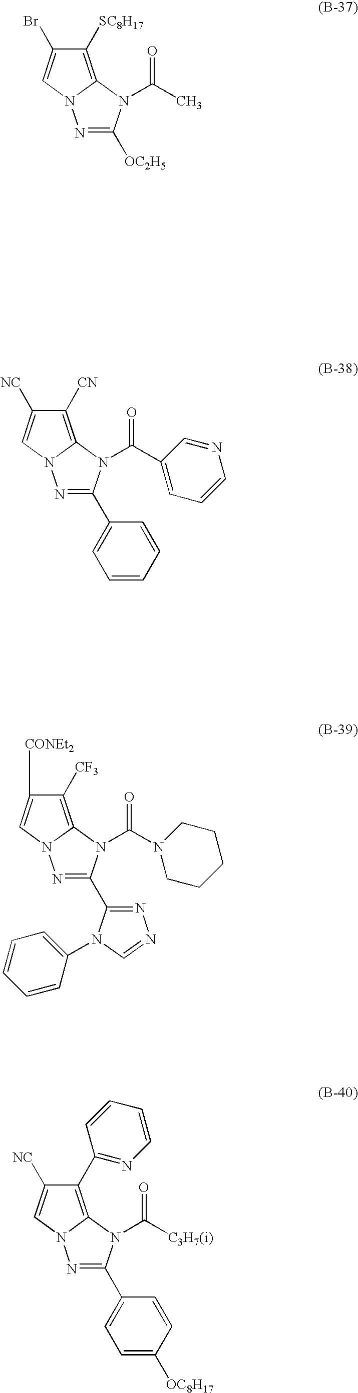

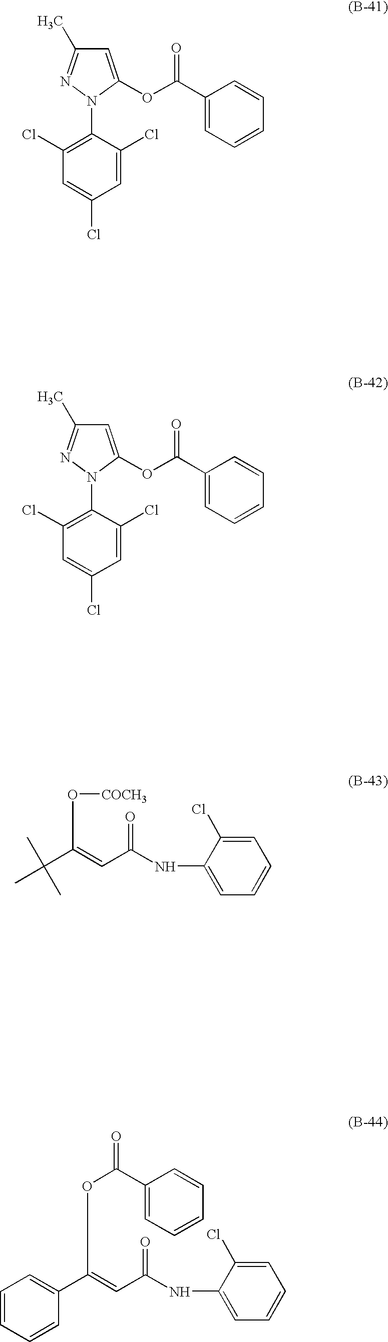

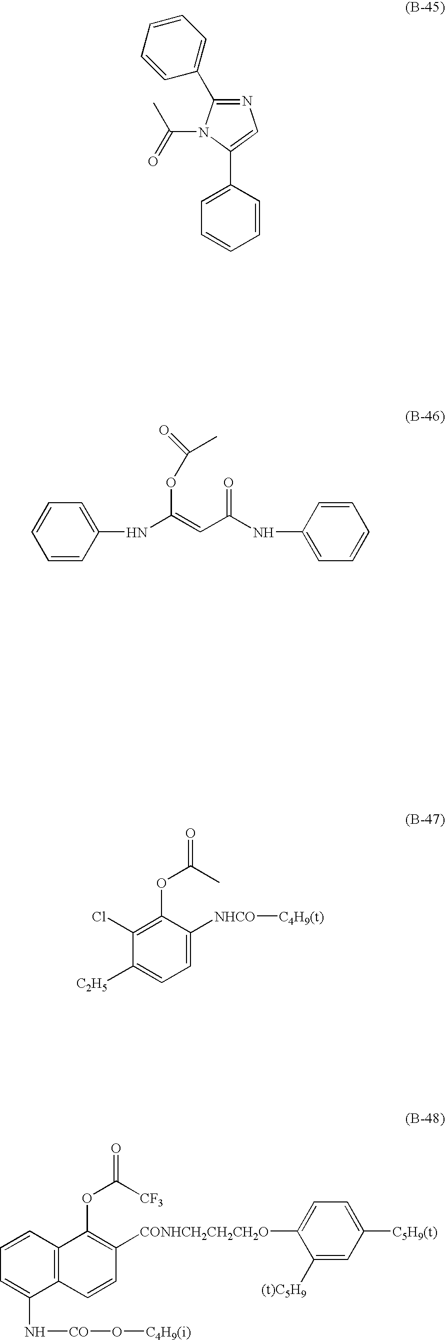

Specific examples of the compound represented by formula (III) are shown below.

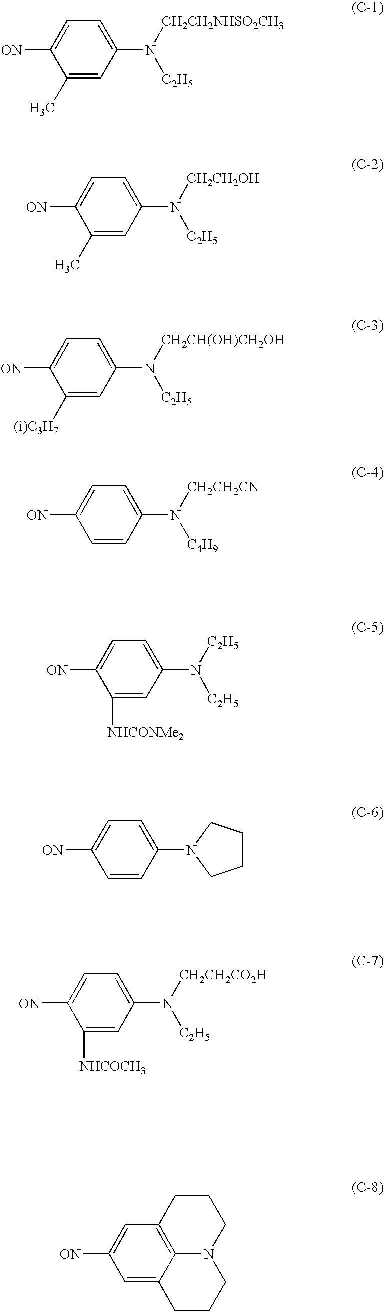

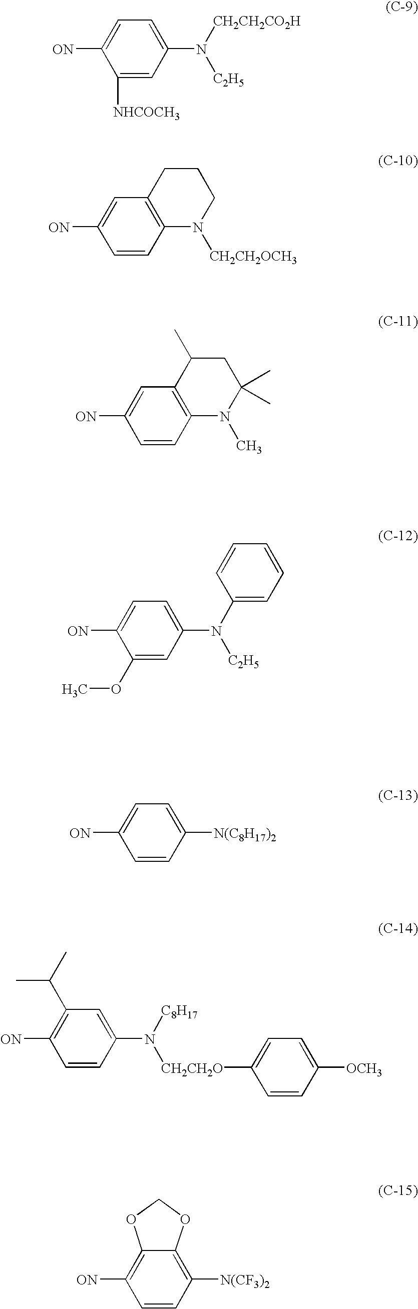

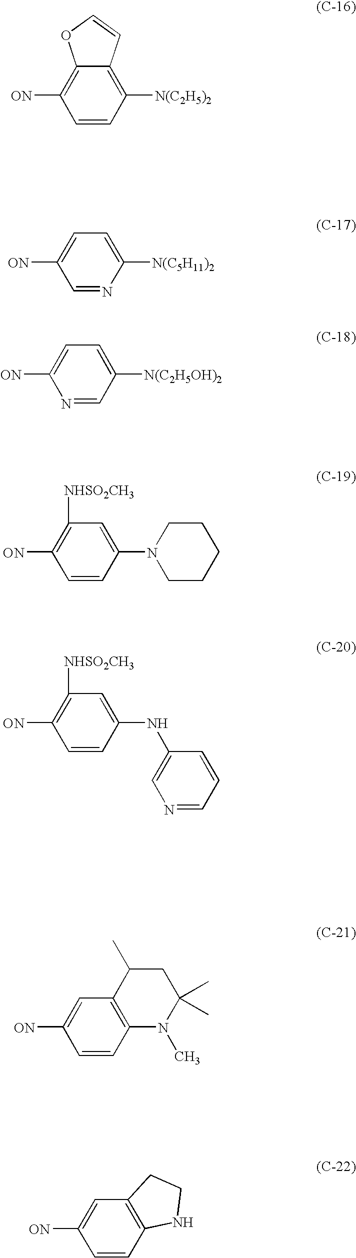

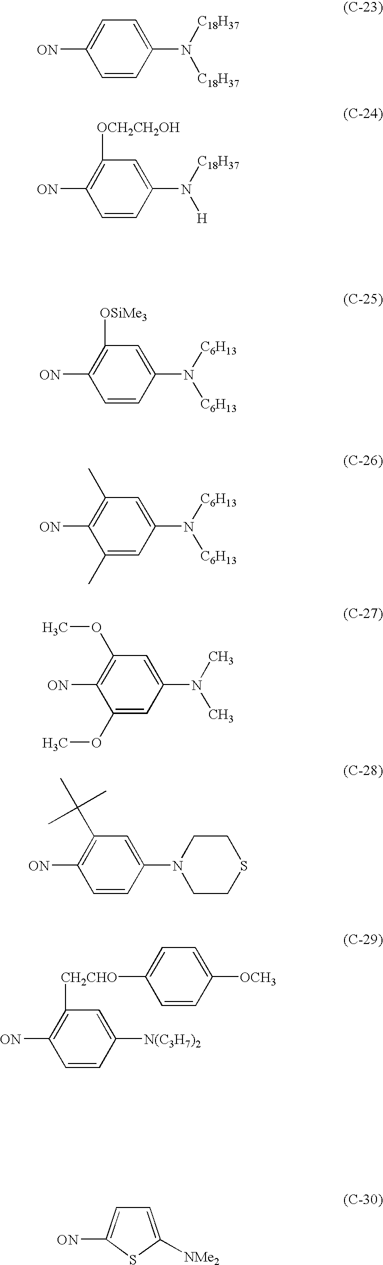

The production method of the present invention is explained below.

The reaction is preferably performed in the presence of a heterocyclic compound containing at least one nitrogen atom. In this connection, this heterocyclic compound differs from the compound represented by any of formulas (I) to (III).

The heterocyclic compound is preferably a nitrogen-containing heterocyclic compound having a five- or six-membered ring, which may further form a condensed ring. The heterocyclic compound is more preferably pyrrole, pyrazole, imidazole, 1,2,4-triazole, tetrazole, indole, pyridine, 2-methylpyridine, 3-methylpyridine, 4-methylpyridine, 2,6-dimethylpyridine, 4-dimethylaminopyridine, 4-pyrrolodinopyridine, 2-chloropyridine, 2-bromopyridine, 3-chloropyridine, 2-cyanopyridine, 2-hydroxypyridine, 2-methoxypyridine, pyridazine, pyrimidine, triazine, quinoline, or purine. The heterocyclic compound is further more preferably pyrrole, pyrazole, imidazole, pyridine, 2-methylpyridine, 3-methylpyridine, 4-methylpyridine, 4-dimethylaminopyridine, 4-pyrrolodinopyridine, 2-chloropyridine, 2-cyanopyridine, or 2-methoxypyridine. The heterocyclic compound is still more preferably pyridine, 2-methylpyridine, 3-methylpyridine, 4-methylpyridine, or 4-dimethylaminopyridine. The heterocyclic compound is still more preferably pyridine, 4-methylpyridine, or 4-dimethylaminopyridine. The heterocyclic compound is most preferably pyridine.

The reaction is also preferably performed in the presence of at least one dehydrating condensing agent. The dehydrating condensing agent is preferably an inorganic dehydrating condensing agent (e.g. an acid anhydride, such as sulfuric anhydride and diphosphorus pentoxide; and an acid chloride, such as thionyl chloride and phosphorus oxychloride), or an organic dehydrating condensing agent (e.g. an acid anhydride, such as acetic anhydride and propionic anhydride; an acid halide, such as acetyl chloride; and a diimide, such as N,N′-dicyclohexylcarbodiimide). Specifically, the dehydrating condensing agent is preferably diphosphorus pentoxide, phosphorus oxychloride, acetic anhydride, propionic anhydride, benzoic anhydride, phthalic anhydride, trifluoroacetic anhydride, or acetyl chloride; more preferably acetic anhydride, propionic anhydride, phthalic anhydride, or acetyl chloride; still more preferably acetic anhydride or propionic anhydride; most preferably acetic anhydride. An absorbent, such as a molecular sieve or an inorganic compound capable of incorporating water as a crystal solvent, such as anhydrous sodium sulfate, is also preferably used as a dehydrating agent.

Concerning the ratio between the amounts of the starting materials used in the reaction, the amount of the compound represented by formula (III) is preferably from 0.1 to 5 moles, more preferably from 0.8 to 3 moles, still more preferably from 0.9 to 2 moles, particularly preferably from 1.0 to 1.8 moles, most preferably from 1.0 to 1.5 moles, based on 1 mole of the compound represented by formula (II). It is also preferred that the aforementioned heterocyclic compound containing at least one nitrogen atom be allowed to coexist; the amount thereof is preferably from 0.01 to 10 moles, more preferably from 0.05 to 5 moles, still more preferably from 0.1 to 3 moles, based on 1 mole of the compound represented by formula (II). It is also preferred that the dehydrating condensing agent be allowed to coexist; the amount thereof is preferably from 0.1 to 10 moles, more preferably from 0.5 to 5 moles, still more preferably from 1.0 to 3 moles, yet more preferably from 1.0 to 2 moles, based on 1 mole of the compound represented by formula (II).

As the solvent for use in the reaction, for example, an amide type solvent (for example, N,N-dimethylformamide, N,N-dimethylacetamide, and 1-methyl-2-pyrrolidone), a sulfone type solvent (for example, sulfolane), a sulfoxide type solvent (for example, dimethylsulfoxide), an ureide type solvent (for example, tetramethylurea), an ether type solvent (for example, dioxane and cyclopentylmethylether), a ketone type solvent (for example, acetone and cyclohexanone), a hydrocarbon type solvent (for example, toluene, xylene, and n-decane), a halogen type solvent (for example, tetrachloroethane and chlorobenzene), an alcohol type solvent (for example, methanol, ethanol, isopropyl alcohol, ethylene glycol, cyclohexanol, and phenol), a pyridine type solvent (for example, pyridine, γ-picoline, and 2,6-lutidine), an ester type solvent (for example, ethyl acetate and butyl acetate), a carboxylic acid type solvent (for example, acetic acid and propionic acid), and a nitrile type solvent (for example, acetonitrile) may be used either singly or in combinations. Among these, an amide type solvent, a sulfone type solvent, an ureide type solvent, an ether type solvent, a halogen type solvent, an alcohol type solvent, a pyridine type solvent, an ester type solvent, a carboxylic acid type solvent, and a nitrile type solvent are preferable; an amide type solvent, an ureide type solvent, an alcohol type solvent, a carboxylic acid type solvent, and a nitrile type solvent are more preferable. The solvent for use in the reaction is preferably N-methylpyrrolidone, N,N-dimethylformamide, N,N-dimethylacetamide, N,N′-dimethylimidazolidinone, methanol, ethanol, isopropyl alcohol, butanol, ethylene glycol, acetic acid, or acetonitrile; more preferably N-methylpyrrolidone, N,N-dimethylformamide, ethanol, isopropyl alcohol, butanol, ethylene glycol, acetic acid, or acetonitrile; most preferably isopropyl alcohol. A mixture of an alcohol type solvent and any other solvent may also be preferred.

The reaction temperature is generally from 0 to 250° C., preferably from 20 to 200° C., more preferably from 50 to 150° C., still more preferably from 70 to 130° C. The reaction time is generally from 5 minutes to 30 hours, preferably from 30 minutes to 20 hours, more preferably from 1 to 10 hours.

For example, the coupler skeleton for use as a dye starting material in the present invention may be produced according to the technique disclosed in each of JP-A-60-186567, JP-A-63-145281, JP-A-64-48862, JP-A-64-48863, JP-A-5-88318, JP-A-5-313324, and JP-A-8-109172, and the patent publications cited therein. In the presence or absence of a base, the dye starting material may be allowed to react with an acid anhydride at a temperature of room temperature (20° C.) to 150° C. or with an acid halide at a temperature of 0° C. to 50° C., so that the compound represented by formula (II) can be easily prepared. In the present invention, the other starting material, the compound represented by formula (III), may be typically produced according to the technique disclosed in JP-A-11-12251 and the patent publications cited therein.

The present invention provides a method of producing azomethine dye compounds useful as image-forming materials for printing, such as inkjet printing and thermal transfer printing; optical recording element materials, optical film materials, and the like. More specifically, the present invention provides a method of producing azomethine dye compounds in high yield under mild conditions without the need of complicated operation.

The present invention will be described in more detail based on the following examples, but the invention is not intended to be limited thereto.

### EXAMPLES

### Example 1

Exemplified compound (A-1) was prepared according to the following scheme.

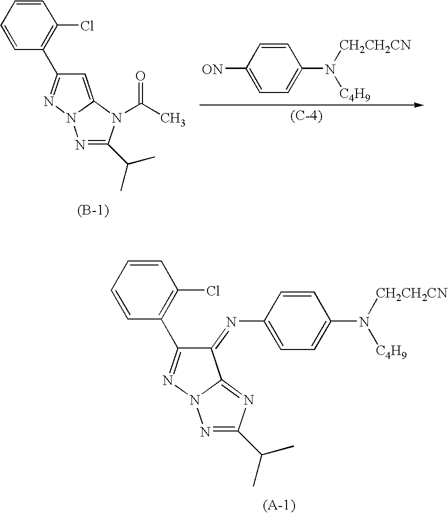

To a three-neck flask were added 30.2 g of Exemplified compound (B-1), 34.7 g of Exemplified compound (C-4), 15.8 g of pyridine, 20.4 g of acetic anhydride, and 120 ml of isopropyl alcohol, and the mixture was heated and stirred under reflux for 6 hours. The resultant was cooled to room temperature, and the deposited crystals were separated by suction filtration and dried to give 45.5 g of the target Exemplified compound (A-1) (yield: 96%). As a result of measurement of the mass spectrum of the product, M⁺ was 473.

NMR (CDCl₃): δ=8.39 (d, J=9.3 Hz, 2H), 7.3-7.7 (m, 4H), 6,78 (d, J=9.3 Hz, 2H), 3.82 (t, J=7.0 Hz, 2H), 3.53 (t, J=7.8 Hz, 2H), 3.25 (qq, J=6.6 Hz, 6.6 Hz, 1H), 2.70 (t, J=7.0 Hz, 2H), 1.3-1.8 (m, 4H), 1.47 (d, J=6.6 Hz, 6H), 1.00 (t, J=7.2 Hz, 3H).

In ethyl acetate, its maximum absorption wavelength λmax was 532.5 nm, and its melting point was 129° C. to 131 ° C.

### Example 2

### Preparation of Exemplified Compound (A-1)

To a three-neck flask were added 30.2 g of Exemplified compound (B-1), 34.7 g of Exemplified compound (C-4), 20.4 g of acetic anhydride, and 120 ml of isopropyl alcohol, and the mixture was heated and stirred under reflux for 10 hours. The resultant was cooled to room temperature, and the deposited crystals were separated by suction filtration and dried to give 31.3 g of the target Exemplified compound (A-1) (yield: 66%).

### Example 3

### Preparation of Exemplified Compound (A-1)

To a three-neck flask were added 30.2 g of Exemplified compound (B-1), 34.7 g of Exemplified compound (C-4), 15.8 g of pyridine, and 120 ml of isopropyl alcohol, and the mixture was heated and stirred under reflux for 11 hours. The resultant was cooled to room temperature, and the deposited crystals were separated by suction filtration and dried to give 34.6 g of the target Exemplified compound (A-1) (yield: 73%).

### Example 4

### Preparation of Exemplified Compound (A-1)

To a three-neck flask were added 30.2 g of Exemplified compound (B-1), 34.7 g of Exemplified compound (C-4), 15.8 g of pyridine, 20.4 g of acetic anhydride, 40 ml of N-methylpyrrolidone, and 80 ml of acetonitrile, and the mixture was heated and stirred under reflux for 6 hours. The resultant was cooled to room temperature. To the resultant were added 500 ml of ethyl acetate and 500 ml of water, and extraction was performed. The resulting ethyl acetate layer was washed with a mixed solution of 300 ml of water and 100 ml of a saturated brine four times, and then concentrated with a rotary evaporator. To the resulting residue was added 250 ml of acetonitrile and stirred. The resulting crystals were separated by suction filtration and dried to give 41.7 g of the target Exemplified compound (A-1) (yield: 88%).

### Example 5

Exemplified compound (A-12) was prepared according to the following scheme.

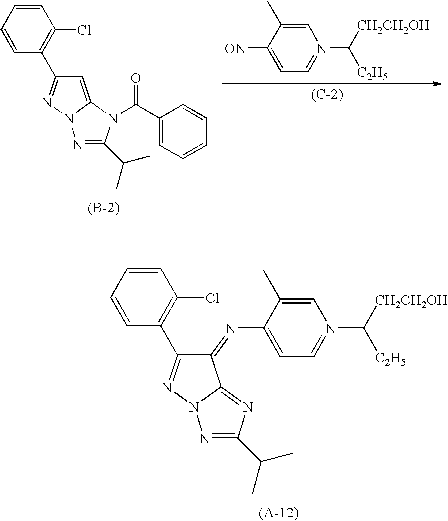

To a three-neck flask were added 36.5 g of Exemplified compound (B-2), 37.5 g of Exemplified compound (C-2), 15.8 g of pyridine, 20.4 g of acetic anhydride, 40 ml of N-methylpyrrolidone, and 80 ml of isopropyl alcohol, and the mixture was heated and stirred under reflux for 8 hours. The resultant was cooled to room temperature. To the resultant were added 500 ml of ethyl acetate and 500 ml of water, and extraction was performed. The resulting ethyl acetate layer was washed with a mixed solution of 300 ml of water and 100 ml of a saturated brine four times, and then concentrated with a rotary evaporator. The resulting residue was purified by silica gel column chromatography to give 37.4 g of the target Exemplified compound (A-12) (yield: 83%). As a result of measurement of the mass spectrum of the product, M⁺ was 450.

### Example 6

Exemplified compound (A-13) was prepared according to the following scheme.

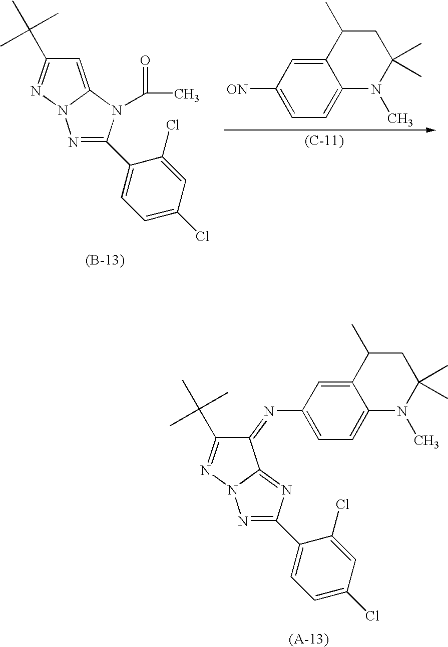

To a three-neck flask were added 35.1 g of Exemplified compound (B-13), 32.7 g of Exemplified compound (C-11), 15.8 g of pyridine, 20.4 g of acetic anhydride, 60 ml of N-methylpyrrolidone, and 60 ml of isopropyl alcohol, and the mixture was heated and stirred under reflux for 6 hours. The resultant was cooled to room temperature. To the resultant were added 500 ml of ethyl acetate and 500 ml of water, and extraction was performed. The resulting ethyl acetate layer was washed with a mixed solution of 300 ml of water and 100 ml of a saturated brine four times, and then concentrated with a rotary evaporator. The resulting residue was purified by silica gel column chromatography to give 39.2 g of the target Exemplified compound (A-13) (yield: 77%). As a result of measurement of the mass spectrum of the product, M⁺ was 508.

### Example 7

Exemplified compound (A-18) was prepared according to the following scheme.

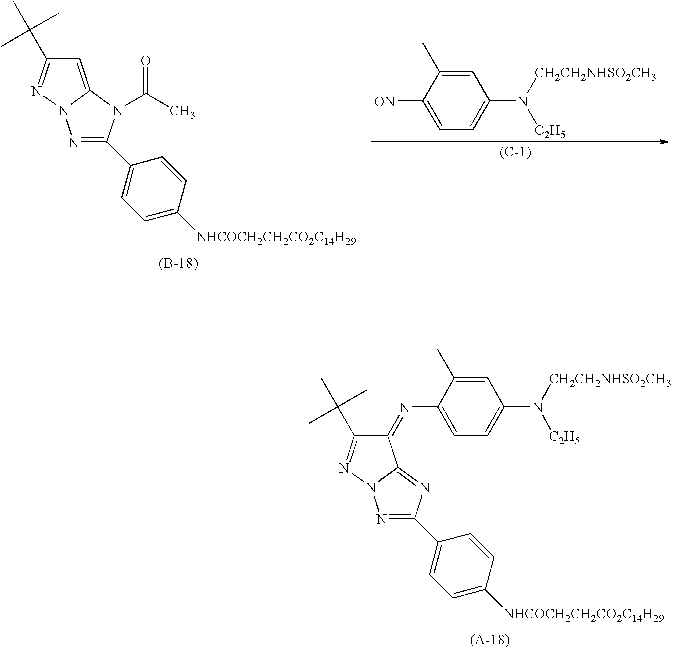

To a three-neck flask were added 59.4 g of Exemplified compound (B-18), 42.8 g of Exemplified compound (C-1), 15.8 g of pyridine, 20.4 g of acetic anhydride, 30 ml of N-methylpyrrolidone, and 90 ml of acetonitrile, and the mixture was heated and stirred under reflux for 6 hours. The resultant was cooled to room temperature. To the resultant were added 500 ml of ethyl acetate and 500 ml of water, and extraction was performed. The resulting ethyl acetate layer was washed with a mixed solution of 300 ml of water and 100 ml of a saturated brine four times, and then concentrated with a rotary evaporator. The resulting residue was purified by silica gel column chromatography to give 65.5 g of the target Exemplified compound (A-18) (yield: 80%). As a result of measurement of the mass spectrum of the product, M⁺ was 818.

### Example 8

Exemplified compound (A-21) was prepared according to the following scheme.

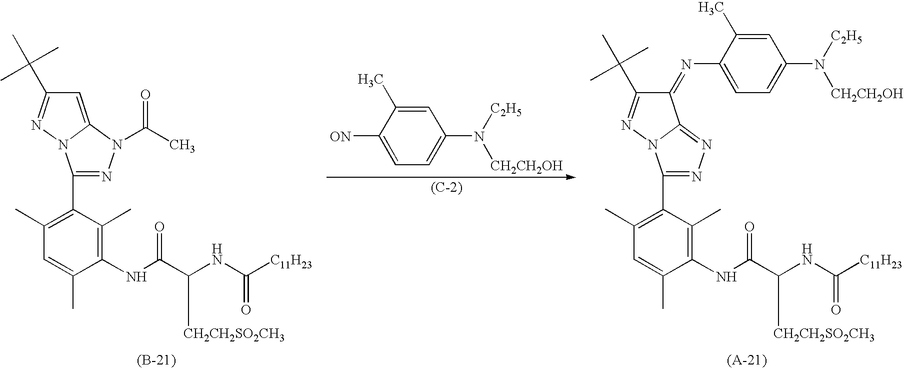

To a three-neck flask were added 68.5 g of Exemplified compound (B-21), 31.2 g of Exemplified compound (C-2), 15.8 g of pyridine, 20.4 g of acetic anhydride, 30 ml of N-methylpyrrolidone, and 90 ml of acetonitrile, and the mixture was heated and stirred under reflux for 6 hours. The resultant was cooled to room temperature. To the resultant were added 500 ml of ethyl acetate and 500 ml of water, and extraction was performed. The resulting ethyl acetate layer was washed with a mixed solution of 300 ml of water and 100 ml of a saturated brine four times, and then concentrated with a rotary evaporator. The resulting residue was purified by silica gel column chromatography to give 59.2 g of the target Exemplified compound (A-21) (yield: 71%). As a result of measurement of the mass spectrum of the product, M⁺ was 832.

### Example 9

### Preparation of Exemplified Compound (A-32)

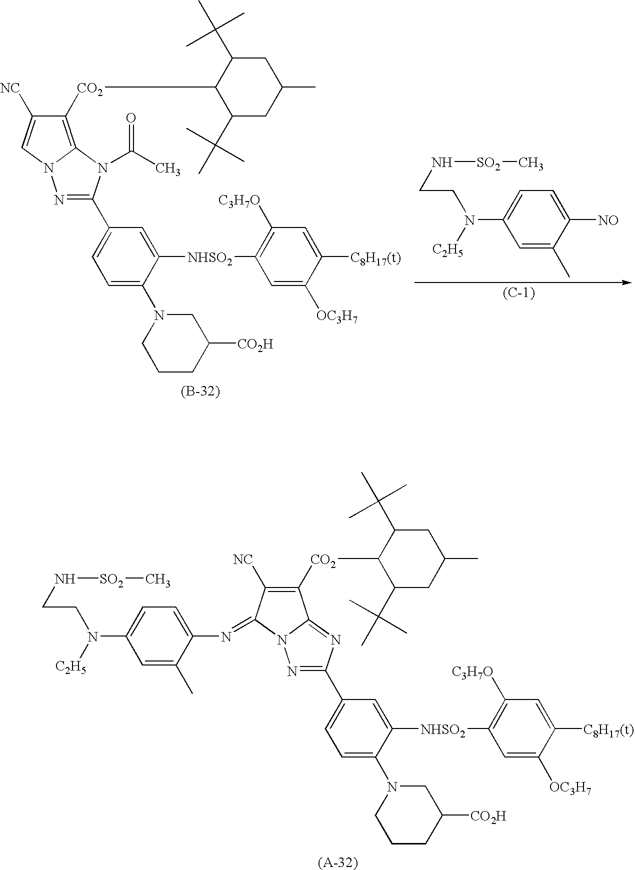

To a three-neck flask were added 101.2 g of Exemplified compound (B-32), 42.8 g of Exemplified compound (C-1), 15.8 g of pyridine, 20.4 g of acetic anhydride, 30 ml of N-methylpyrrolidone, and 90 ml of acetonitrile, and the mixture was heated and stirred under reflux for 8 hours. The resultant was cooled to room temperature. To the resultant were added 500 ml of ethyl acetate and 500 ml of water, and extraction was performed. The resulting ethyl acetate layer was washed with a mixed solution of 300 ml of water and 100 ml of a saturated brine four times, and then concentrated with a rotary evaporator. The resulting residue was purified by silica gel column chromatography to give 83.0 g of the target Exemplified compound (A-32) (yield: 67%). As a result of measurement of the mass spectrum of the product, M⁺ was 1,237.

In ethyl acetate, its maximum absorption wavelength λmax was 641.3 nm,

### Example 10

### Preparation of Exemplified Compound (A-41)

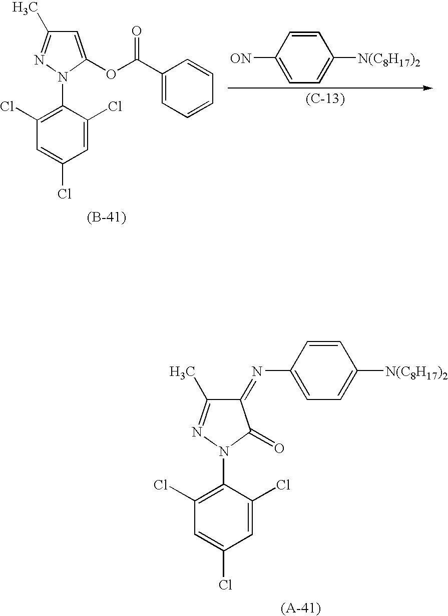

To a three-neck flask were added 38.2 g of Exemplified compound (B-41), 52.0 g of Exemplified compound (C-13), 15.8 g of pyridine, 20.4 g of acetic anhydride, 60 ml of N-methylpyrrolidone, and 60 ml of acetonitrile, and the mixture was stirred at an inner temperature of 90° C. for 6 hours. The resultant was cooled to room temperature. To the resultant were added 500 ml of ethyl acetate and 500 ml of water, and extraction was performed. The resulting ethyl acetate layer was washed with a mixed solution of 300 ml of water and 100 ml of a saturated brine four times, and then concentrated with a rotary evaporator. The resulting residue was purified by silica gel column chromatography to give 40.6 g of the target Exemplified compound (A-41) (yield: 67%). As a result of measurement of the mass spectrum of the product, M⁺ was 604.

Having described our invention as related to the present embodiments, it is our intention that the invention not be limited by any of the details of the description, unless otherwise specified, but rather be construed broadly within its spirit and scope as set out in the accompanying claims.

What we claim is:

1. A method of producing a compound represented by formula (I), comprising reacting a compound represented by formula (II) with a compound represented by formula (III): 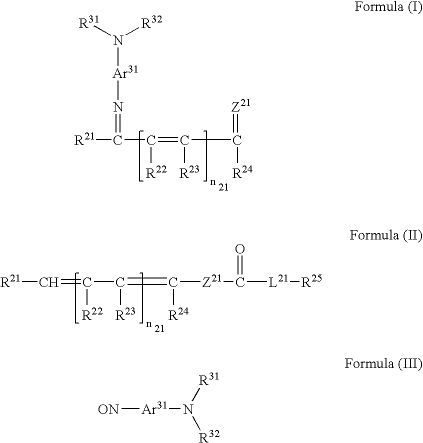
wherein, in formulas (I) to (III), Ar³¹ represents a divalent aromatic group or a divalent heterocyclic group; L²¹ represents a single bond, —O—, or —N(R²⁶)—; Z² represents —O— or —N(R²⁷)—; R²¹, R²², R²³, R²⁴, and R²⁷ each independently represent a hydrogen atom or a substituent; R²⁵, R²⁶, R³¹, and R³² each independently represent a hydrogen atom, an aliphatic group, an aromatic group, or a heterocyclic group bonding through its carbon atom; and n₂₁ represents 0 or 1, wherein any of R²¹, R²², R²³, R²⁴, and R²⁷ may bond to one another to form a ring, and any of Ar³¹, R³¹, and R³² may bond to one another to form a ring.

2. The method as claimed in claim 1, wherein the compound represented by formula (I) is a compound represented by formula (I-1) or (I-2), the compound represented by formula (II) is a compound represented by formula (II-1) or (II-2), and the compound represented by formula (III) is a compound represented by formula (III-1): 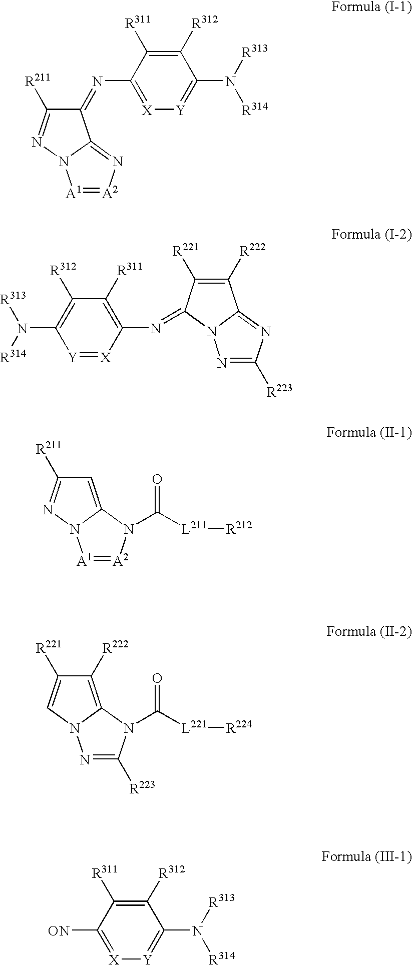
wherein, in formulas (I-1), (I-2), (II-1), (II-2), and (III-1), L²¹¹ and L²²¹ each independently represent a single bond, —O—, or —N(R²¹³)—; one of A¹ and A² represents a nitrogen atom and the other represents —C(R²¹⁴)═; X and Y each independently represent a nitrogen atom or ═C(R³¹⁵)—; R²¹¹, R²¹⁴, R²²¹, R²²², R²²³, R³¹¹, R³¹², and R³¹⁵ each independently represent a hydrogen atom or a substituent; and R²¹², R²¹³, R²²⁴, R³¹³, and R³¹⁴ each independently represent a hydrogen atom, an aliphatic group, an aromatic group, or a heterocyclic group bonding through its carbon atom.

3. The method as claimed in claim 1, wherein the reaction is carried out in the presence of a heterocyclic compound containing at least one nitrogen atom, and the heterocyclic compound differs from the compounds represented by formulas (I) to (III).

4. The method as claimed in claim 3, wherein the heterocyclic compound containing at least one nitrogen atom is used in an amount of 0.01 to 10 moles per mole of the compound represented by formula (II).

5. The method as claimed in claim 3, wherein the heterocyclic compound containing at least one nitrogen atom is pyrrole, pyrazole, imidazole, 1,2,4-triazole, tetrazole, indole, pyridine, 2-methylpyridine, 3-methylpyridine, 4-methylpyridine, 2,6-dimethylpyridine, 4-dimethylaminopyridine, 4-pyrrolodinopyridine, 2-chloropyridine, 2-bromopyridine, 3-chloropyridine, 2-cyanopyridine, 2-hydroxypyridine, 2-methoxypyridine, pyridazine, pyrimidine, triazine, quinoline, or purine.

6. The method as claimed in claim 1, wherein the reaction is carried out in the presence of a dehydrating condensing agent.

7. The method as claimed in claim 6, wherein the dehydrating condensing agent is used in an amount of 0.1 to 10 moles per mole of the compound represented by formula (II).

8. The method as claimed in claim 6, wherein the dehydrating condensing agent is diphosphorus pentoxide, phosphorus oxychloride, acetic anhydride, propionic anhydride, benzoic anhydride, phthalic anhydride, trifluoroacetic anhydride, or acetyl chloride.

9. The method as claimed in claim 1, wherein the method produces a dye compound.

10. The method as claimed in claim 1, wherein the compound represented by formula (III) is used in an amount of 0.1 to 5 moles per mole of the compound represented by formula (II).
